(19) United States
(12) Patent Application Publication
(10) Pub. No.: US 2026/0245690 A1
(43) Pub. Date: Aug. 20, 2026

(54) AUTOMATED WORKFLOW FOR SOCIAL DETERMINANTS OF HEALTH DATA MANAGEMENT

(71) Applicant: Alvee Health Inc., Miami, FL (US)

(72) Inventor: Nicole Cook, Miami, FL (US)

(73) Assignee: Alvee Health Inc., Miami, FL (US)

(21) Appl. No.: 19/056,603

(22) Filed: Feb. 18, 2025

(51) Int. Cl. G16H 20/00; G06N 20/00; G16H 10/60; G16H 70/60

(52) U.S. Cl. CPC G16H 20/00; G06N 20/00; G16H 10/60; G16H 70/60

(57) ABSTRACT

The technology disclosed teaches a system and methods for generating a personalized care plan based on social determinants of health. The method further comprises pre-processing unstructured patient data corresponding to a patient to generate structured patient data and processing the structured patient data using a machine learning model, wherein the machine learning model is pre-trained to generate output data including at least one of a barrier to care, a disease risk factor, a discrepancy in the structured patient data, a risk score, and a recommended SDoH intervention. The method further includes creating a personalized care plan for the patient, based on the output data, including a personalized resource recommendation, wherein the personalized resource recommendation identifies an action plan responsive to an identified barrier to care.

### FIELD OF THE TECHNOLOGY DISCLOSED

The technology disclosed relates generally to an artificial intelligence-driven workflow automation and data management platform that enables providers and payers to reduce health disparities, advance health equity, advance health outcomes for patients, assist with regulatory compliance and improve cost efficiency. The technology disclosed further relates to an integrated tool platform that provides, to both care providers and patient navigators, a comprehensive view of a patient's health equity status by incorporating social determinants of health, chronic conditions, demographic information, and health inequity risks, thereby enabling payers and providers to meet data stratification quality reporting requirements.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves may also correspond to implementations of the claimed technology.

Social determinants of health (SDoH) (i.e., the circumstances in which people are born, live, learn, work, and age) affect a wide range of health and function outcomes, quality of life (QoL), and risks that are closely tied to individuals' health behaviors, lifestyle, and interpersonal relationships. These environmental and behavioral factors can impede disease management and may further lead to (or exacerbate) existing comorbid conditions. Health systems have been increasingly attuned to these detriments given their impact on healthcare outcomes and costs. The strong association between nonclinical factors (e.g., socioeconomic status) and clinical outcomes have increased clinical and public health interests in integrating SDoH into patient profiles on a broad scale. Incorporation of SDoH into disease models for screening and prediction can inform identification of various social risks. SDoH factors can account for enormous variability in health outcomes that are modifiable via clinical and social interventions and are thus promising targets for healthcare initiatives.

The collection and analysis of SDoH information can offer significant advantages including discovery of valuable insights about patient lifestyle, which can in turn be used to augment clinical findings. Understanding the impact of SDoH factors can further empower providers to consider and address underlying determinants of health that contributes to poor health outcomes and health disparities, and consequently develop intervention strategies to address potential risks and solutions for optimal patient-centered care. The digitization of clinical records improves the feasibility and scalability of SDoH integration into patient records (e.g., via electronic health records (EHR)) to enhance healthcare delivery.

An opportunity arises to provide a comprehensive view of a patient's health equity status by incorporating SDoH, chronic conditions, demographic information, and health inequity risks, enabling payers and providers to meet data stratification quality reporting requirements. An opportunity also arises for an AI-driven workflow automation and data management SaaS platform that enables providers and payers to reduce health disparities, advance health equity, advance health outcomes for patients, assist with regulatory compliance and improve cost efficiency.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings.

FIG. 1 is a schematic diagram illustrating generation of an end-to-end social determinants of health (SDOH) management system.

FIG. 2A is a flow diagram illustrating an example of generating a personalized care plan from patient data.

FIG. 2B is a flow diagram illustrating an example of generating a personalized care plan from patient data.

FIG. 2C is a flow diagram illustrating an example of generating a personalized care plan from patient data.

FIG. 2D is a flow diagram illustrating an example of generating a personalized care plan from patient data.

FIG. 3 is an architectural diagram of an autonomous care navigator.

FIG. 4A is an architectural diagram of a data pre-processor, according to some implementations of the technology disclosed.

FIG. 4B shows an example workflow for the pre-processing of patient data, according to some implementations of the technology disclosed.

FIG. 5 is a schematic diagram for fine-tuning a pre-trained machine learning model.

FIG. 6 is an architectural diagram of an ICD-10 Z-Code Transformer for SDoH data standardization, according to some implementations of the technology disclosed.

FIG. 7 is a schematic diagram depicting an example algorithm for extracting SDoH-related terminology from patient data.

FIG. 8 is a schematic diagram showing vector embedding of SDoH data.

FIG. 9 is an architectural diagram of a SaaS platform that can be leveraged in clinical decision-making.

FIG. 10 is an example GUI dashboard associated with the SaaS platform of FIG. 9.

FIG. 11 is a block diagram of a process for generating information and presentation of generated information to a user via the GUI of FIG. 10.

FIG. 12 is a second example GUI associated with the SaaS platform of FIG. 9.

FIG. 13 is an example of a computer system architecture implementing the disclosed autonomous care navigator.

### DETAILED DESCRIPTION

The following detailed description is made with reference to the figures. Sample implementations are described to illustrate the technology disclosed, not to limit its scope, which is defined by the claims. Those of ordinary skill in the art will recognize a variety of equivalent variations on the description that follows.

### Acronyms

Acronyms used in this disclosure are identified the first time that they are used. These acronyms are terms of art. Except where the terms are used in a clear and distinctly different sense than they are used in the art, we adopt the meanings found in the art. For the reader's convenience, some of them are listed next.

| | |
|---|---|
| ACSC | Ambulatory Care Sensitive Condition |
| AI | Artificial Intelligence |
| API | Application Programming Interface |
| ASIC | Application-Specific Integrated Circuit |
| CDA | Clinical Document Architecture |
| C-CDA | Consolidated Clinical Document Architecture |
| CHEAR | Child Health Exposure Analysis Resource |
| CNN | Convolutional Neural Network |
| CPT | Current Procedural Terminology |
| CRM | Customer Relationship Management |
| DNN | Deep Neural Network |
| DSP | Digital Signal Processor |
| ECTO | Environment Conditions, Treatments, |
| | and Exposures Ontology |
| EHR | Electronic Health Records |
| EMR | Electronic Medical Records |
| ENVO | Environment Ontology |
| FPGA | Field Programmable Gate Array |
| FPOA | Field Programmable Object Array |
| GNN | Graph Neural Network |
| GUI | Graphical User Interface |
| HCPCS | Healthcare Common Procedure Coding System |
| HHEAR | Human Health Exposure Analysis Resource |
| ICD | International Classification of Diseases |
| ICD-10 | 10^{th} Revision of the International |
| | Classification of Diseases |
| LLM | Large Language Model |
| LOINC | Logical Observation Identifiers Names and Codes |
| LSTM | Long Short-Term Memory |
| NDC | National Drug Code |
| NER | Named Entity Recognition |
| NLP | Natural Language Processing |
| OMRSE | Ontology of Medically Related Social Entities |
| PLA | Programmable Logic Array |
| QoL | Quality of Life |
| RAG | Retrieval-Augmented Generation |
| RLHF | Reinforcement Learning with Human Feedback |
| RNN | Recurrent Neural Network |
| SaaS | Software as a Service |
| SDOH | Social Determinants of Health |
| SMASH | Semantic Mining of Activity, Social, and Health |
| SME | Subject Matter Expert |
| SNOMED | Systemized Nomenclature of Medicine Clinical Terms |
| USCDI | United States Core Data for Interoperability |

### Overview of the Technology Disclosed

According to The Healthy People 2030 Initiative, developed by the US Department of Health and Human Services, Social Determinants of Health (SDoH) are circumstances in which people are born, live, learn, work, and age that influence health status and outcomes. SDoH affect a wide range of health, functioning, quality of life (QoL) outcomes, and risks that are closely tied to individuals' health behaviors, lifestyle, and interpersonal relations. Environmental and behavioral factors associated with SDoH, such as socioeconomic status, education, social supports, and gender, can impede disease self-management, induce disease states, or exacerbate existing comorbid conditions. Health systems have become progressively more attuned to SDoH given their impact as upstream drivers of poor health outcomes and higher healthcare costs. Clinical and public health interests in SDoH have increased exponentially in response to a growing body of relationships identified between nonclinical, environmental, and/or behavioral factors. In practice, SDoH are being incorporated into patient profiles and disease models on an increasingly broadened scale.

As the volume of existing SDoH data grows, so does a body of evidence suggesting that social determinants can account for enormous variability in health outcomes, i.e., disparities as measured by quantitative factors including statistical significance and magnitude of scale. Decades of health equity research demonstrate that individuals or populations who have been deprived of certain SDoH (e.g., wealth and social privilege) are disadvantaged by health disparities and face worse healthcare outcomes than those with access to said certain SDoH. However, many of the health outcomes affected by SDoH inequities are modifiable outcomes. For example, individuals that live in neighborhoods with limited access to fresh foods (“food deserts”) are at higher risk for a range of adverse health outcomes. Nutritional access can be modified by public health intervention, which can in turn improve outcomes and reduce inequities. Consequently, SDoH are frequently key targets for intervention.

Collection and analysis of SDoH data can offer valuable patient insights that can be applied to many subsectors of the healthcare industry, including research & development, public health intervention, and clinical implementation. A better understanding of social determinants, as well as how to effectively address the corresponding impact of a social determinant, leads to a myriad of benefits including an improvement to societal health at-scale, public health and research initiatives that are better informed and more efficacious, higher quality patient care, streamlined operations within the healthcare industry, and mitigation of costs.

The digitization of clinical records presents a new opportunity for enhanced healthcare delivery vis-à-vis integration of SDoH into EHR. Unfortunately, difficult challenges prevail within navigation workflows concerning SDoH integration. Validation research conducted by the Applicant and external partners exploring needs for innovative technology solutions in SDoH integration revealed crucial pain points experienced by healthcare systems and care managers. Existing barriers that lack feasible solutions include resource deficiencies, data gaps, interoperability issues, staff burnout, and limited efficiency in resource discovery. Healthcare organizations and clinics grapple with limited resources and manpower necessary to efficiently monitor SDoH data. Data collection relating to SDoH often contains larger gaps than other forms due to a lack of standardization and human errors. Furthermore, the inconsistencies and gaps in data can skew data quality for analysis purposes. EHR platforms are generally not standardized across different systems for SDoH, which additionally complicates data analysis. Clinical networks and offices may use different EHR platforms from one another, and in combination with differences in data collection and formatting between these different platforms, these circumstances can limit both the quantity and quality of available data. Care managers and other healthcare professionals consequently need to perform tedious manual collection and analysis of SDoH data from different patient charts and data sources, which contributes significantly to staff burnout. Care managers and social workers invest substantial time into manually curating personalized lists of resources for patients, limiting scalability and consistency of such practices. The risk of introducing bias and error progressively worsens across the SDoH data collection and analysis process as the impact of data quality, lack of standardization, and reliance on manual labor compound one another.

Inefficiencies in the data collection and analysis pipeline damage the feasibility of useful, meaningful SDoH integration at scale in terms of operational efficiency (e.g., the resource barriers discussed above) and prohibitive costs. According to American Hospital Association reports, annual financial losses for the American healthcare system are estimated at approximately $135B, in large part due to excess costs of care and untapped productivity. The sheer size of many healthcare systems can make it difficult to address operational inefficiency. Unfortunately, the present reality is that many healthcare organizations fail to prioritize identifying and remedying sources of operational inefficiency because such processes can be expensive, time-consuming, and lack a guaranteed return on investment. Healthcare focuses that are more progressive, or in earlier stages of development, like SDoH integration are often heavily impacted by these leadership decisions despite their potential to boost worker productivity, reduce health disparities, decrease financial loss, and improve compliance with requirements by the government and licensure bodies.

The movement to address Social Determinants of Health (SDoH) and healthcare disparities has gained significant momentum across the healthcare ecosystem in recent years. While federal policies may shift with each administration, agencies including the U.S. Department of Health and Human Services (HHS), Department of Agriculture (USDA), Department of Housing and Urban Development (HUD), Department of Veterans Affairs (VA), and the Environmental Protection Agency (EPA) have introduced impactful SDoH-related initiatives, many of which are reinforced by ongoing efforts within healthcare networks, payers, and state and local agencies. Beyond regulatory mandates, the industry-wide push for value-based care and improved health outcomes continues to drive investment in SDoH integration. However, ensuring compliance with evolving regulations remains a challenge, as existing frameworks are often not structured to support seamless SDoH data integration. To meet modern requirements and avoid penalties, organizations must demonstrate their ability to accurately acquire, maintain, and report health data while effectively addressing health disparities.

As such, a need exists for innovative solutions for SDoH integration into EHR systems that empower healthcare organizations to remain compliant with changing expectations while minimizing undue burden. Important solutions include: prioritization strategies to address barriers to care and social risks on a patient-by-patient basis; standardization of SDoH data including coding of social needs into ICD-10 Z-Codes; generation of patient-specific precision interventions using predictive analytics; development of predictive models with real-world capacity for multiple data input channels; approaches for supplementing data with missing information and otherwise addressing data gaps; consistent identification of accessible resources; seamless incorporation of resource access and intervention into personalized care plans; and automation of labor-intensive tasks within all aspects of a comprehensive SDoH-integrated framework.

System and method implementations of the technology disclosed provide the solutions listed above via the use of workflow automation and SDoH data management for efficient clinical decision-making and improved patient health outcomes. One implementation of the technology disclosed relates to a method for generating a personalized care plan for a patient based on SDoH. The method comprises pre-processing unstructured patient data corresponding to a patient, received from a plurality of disparate patient data sources. The pre-processing operation includes parsing the unstructured patient data for clinically relevant information and extracting features within the parsed clinically relevant information. At least one extracted feature is based on supplemental data from a clinical database, a SDoH dataset, or a SDoH coding dictionary. The pre-processing further includes converting the unstructured patient data into structured patient data that includes the extracted features. The method also includes processing the structured patient data using a SDoH machine learning model, wherein the SDoH machine learning model is pre-trained to generate SDoH output data including at least one of a barrier to care, a disease risk factor, a discrepancy in the structured patient data, a risk score, and/or a recommended SDoH intervention. The method further includes creating a personalized care plan for the patient, based on the SDoH output data, including a personalized resource recommendation, wherein the personalized resource recommendation: (i) identifies an action plan responsive to an identified barrier to care, and (ii) is constrained by a set of rules limiting the personalized resource recommendation to resources that are compatible with a geographic location, a socioeconomic status, or a disability status of the patient. The method further includes displaying the personalized care plan to a user via a user interface of an autonomous care navigator. The user interface is configured to receive user feedback including updated patient data or progress data related to execution of the personalized care plan. The method further includes updating the personalized care plan based on the user feedback, a feedback loop for the SDoH machine learning model leverages the user feedback to refine the SDoH output data, and the personalized care plan is updated in dependence on the refined SDoH output data.

Many implementations of the technology disclosed comprise an autonomous care navigator to generate of one or more personalized SDoH care plans. In certain implementations, the autonomous care navigator further comprises an AI agent such as a generative AI agent. Various implementations of the disclosed system further comprise a platform to generate health equity alerts. In one example implementation, the AI agent is integrated within the health equity alert platform via an application programming interface (API). The autonomous care navigator can also operate in association with the health equity alert platform, potentially in further combination with one or more database systems such as EHR, electronic medical records (EMR), and/or customer relationship management (CRM) databases.

Other implementations of the technology disclosed relate to methods for producing a patient-specific and longitudinally adaptable SDoH care plan. Many disclosed methods comprise the autonomous care navigator receiving health data for a patient, identifying one or more barriers to care for the patient based on the health data, selecting a prioritized barrier to care of the one or more identified barriers to care, automatically capturing a clinical code corresponding to the prioritized barrier to care, and standardizing the captured clinical code. Other disclosed methods comprise automatically capturing a reimbursement-related charge associated with the patent. Another method comprises the autonomous care navigator identifying one or more social risk for the patient based on the health data, selecting a prioritized social risk of the one or more identified social risks, identifying one or more resources corresponding to the prioritized social risk, and developing a SDoH care plan based on the one or more identified resources. Yet other methods disclosed comprise integrating patient-specific social environment factors and clinically relevant determinants of health into the SDoH care plan. In some implementations, the autonomous care navigator evaluates patient data to determine statistical associations between a barrier to care and a health risk. Many implementations of the technology disclosed relate to producing a patient-specific, longitudinally adaptable SDoH care plan that is formatted for seamless integration with EHRs. In the interest of conciseness, alternative combinations of method operations are not individually enumerated. The reader will understand how features identified in one particular disclosed method can readily be combined with base features in other disclosed methods.

Various implementations of the technology disclosed include a multi-faceted ensemble strategy for generating a customizable SDoH care plan. The multi-faceted ensemble can include a combination of SDoH data operations including SDoH data sourcing, collecting, curating, preprocessing, annotating, cleaning, augmenting, transforming, and learning using a machine learning method. The SDoH data can be obtained from a single source of multiple sources, such as an EHR database or clinical trial data. The SDoH data can further be obtained from internal and/or external data sources. Various features included in the SDoH data can include, for example, patient demographic, geolocation, International Classification of Diseases (ICD) code, Current Procedural Terminology (CPT) code, Healthcare Common Procedure Coding System (HCPCS) code, National Drug Code (NDC), Logical Observation Identifiers Names and Codes (LOINC), Systematized Nomenclature of Medicine Clinical Terms SNOMED, among others which are readily recognizable to a skilled user, located within an internal or external EMR/EHR database. The innovative data collection and management solutions offered by the technology disclosed will now be discussed in further detail.

### Solutions for Patient Data Collection and Management

The technology disclosed provides a solution that fulfills the long felt need for patient data processing tools that are adaptive to unstructured and/or unstandardized data. Herein, the term “unstructured patient data” refers to any data that includes at least one source of unstructured data. The unstructured patient data may include entirely unstructured data, such as free, unformatted text written by a user (e.g., a primary care provider) or transcribed from a phone call. Frequently, the unstructured patient data includes a combination of unstructured patient data as well as structured patient data, wherein structured patient data can include extracted EMR/EHR information structured into pre-defined fields with a constrained format (e.g., an ethnicity field wherein the field value is one category selected from a predetermined list of a fixed number of categories) or pre-cleaned data extracted from a relational database. In many cases, the unstructured patient data is “mixed structure” patient data, wherein mixed structure refers to a lack of standardized structure or formatting across the plurality of data sources.

For example, first unstructured patient data corresponding to a first client may include data accumulated from a Source A, Source B, and Source C. Source A includes completely unstructured patient data, e.g., text with no pre-defined fields or constraint on formatting, like social worker notes. Source B includes partially structured patient data, e.g., a combination of some unstructured text data and certain data attributes organized into structured fields, such as a Patient Visit Summary that includes laboratory test results in a tabular format and clinician discharge instructions in an unstructured format. Source C includes entirely structured data, such as curated demographic data presented as encoded vectors. Consequently, Sources A, B, and C do not have standardized formatting across data that would allow easily merging data across sources. Furthermore, second unstructured patient data corresponding to a second client may include data accumulated from a Source D, Source E, and Source F. Analogously to Source A, Source B, and Source C, each of Source D, Source E, and Source F can respectively contain unstructured, structured, or mixed structure data. Additionally, Source D, Source E, and Source F will contain at least some portion of non-overlapping data content and structure such that the three data sources are dissimilar to one another, much like Source A, Source B, and Source C are dissimilar to one another. Moreover, one or more of Source A, Source B, and Source C corresponding to the first patient is dissimilar to one or more of Source D, Source E, and Source F. The first unstructured patient data may include, as mentioned above, social work notes, patient visit summaries, and demographic data. The second unstructured patient data can include medication history, a qualitative patient survey, and a problem list of Z-codes. In another example, the first unstructured patient data may include EMR/EHR data obtained from a first medical record software platform while the second unstructured patient data may include EMR/EHR data obtained from a second medical record software platform. The data from each respective software platform will include very similar information, but the information will be structured in different ways and may describe the same information using different attribute labels, different terminology, and/or different encoding protocols.

While the above example of first and second sets of patient data sourced variously from Sources A-F is purely illustrative, said example is representative of a common problem impacting the efficiency and accuracy of patient care management. Patient data, compared to many other data sources, is exceptionally data-rich in volume and variety. As data complexity increases, so does the difficulty of analysis and risk of error. Patient-to-patient variability in demographics, health conditions, SDoH, treatment history, etc., makes it infeasible to restrict or standardize the types of attributes or variables that are tracked for the purpose of data analysis.

As a representative non-limiting example, the ICD-10 coding schema includes over 70,000 codes, some of which may be very commonly used (e.g., codes relating to cardiovascular disease) and some of which are rarely used (e.g., rare recessive-autosomal genetic disorders). From a data science standpoint, it may initially appear pragmatic to exclude “outlier” codes (e.g., a code corresponding to a medical condition that occurs at a rate lower than a pre-defined standpoint) in order to improve overall accuracy and consistency of analysis. From a healthcare standpoint, however, this restrictive strategy considerably limits utility in-practice. Limiting analysis to common ICD codes leads to exclusion of medically complex patients, and medically complex patients are the population most likely to benefit from healthcare data analytics. Furthermore, certain redundancies in the ICD coding system exist that may frustrate data analysis. For example, certain ICD codes pertaining to arthritic disease include:

| | |
|---|---|
| M15.0 | Primary generalized osteoarthritis |
| M15.8 | Other polyosteoarthritis |
| M15.9 | Polyosteoarthritis, unspecified |
| M19.0 | Primary osteoarthritis of other joints |
| M19.90 | Unspecified osteoarthritis, unspecified site |
| M19.91 | Primary osteoarthritis, unspecified site |
| M13.0 | Polyarthritis, unspecified |
| M13.10 | Monoarthritis, not elsewhere classified, unspecified site |
| M13.8 | Other specified arthritis, unspecified site |
| M16.0 | Bilateral primary osteoarthritis of hip |
| M16.2 | Bilateral osteoarthritis resulting from hip dysplasia |

The apparent redundancy of the above coding examples is intentional, and useful in many contexts. The ICD coding system is designed to capture a breadth of data, leading to many similar codes. For example, M19.0 is a nonbillable code while M19.90 is a billable code. Codes that indicate unspecified data allow for clinical staff to document patient data, even if certain information is not available. Ideally, these codes would be used in an unambiguous, consistent way across providers. However, in practice, factors such as ambiguity, nuance, error, failure to update patient data, and environment-specific considerations prevent diagnostic coding from achieving total consistency.

In one illustrative example, consider a patient who has osteoarthritis in both hips. One provider may opt to add M13.0 Polyarthritis, unspecified to a patient's chart because the patient has arthritis in multiple joints, while another provider may use M15.0 Primary generalized osteoarthritis to describe the same patient based on the same rationale. In an emergency department setting, it is less likely that specific details will be available or prioritized if a diagnosis is not related to the presenting emergency. Hence, the emergency medicine provider may simply document the patient's arthritis as M13.8 Other specified arthritis, unspecified site, because they are unaware of the fact that the patient's primary care physician has already documented a diagnosis of M16.0 Bilateral primary osteoarthritis of hip. Furthermore, the patient's rheumatologist may have previously documented a more specific diagnosis than the primary care physician has documented (e.g., M16.2 Bilateral osteoarthritis resulting from hip dysplasia) due to the rheumatologist's specialty and direct care of the patient's osteoarthritis. Similar variability can simply arise from a patient misreporting medical history, a data entry error, failure to update diagnoses as more specific details become available, and so on.

Often, variability in documentation is difficult to catch because the providers work for different organizations and hence, do not have shared access to records of other organizations. Perhaps the primary care physician owns a private practice, the emergency department is operated by one hospital network, and the rheumatologist is affiliated with a different hospital network. However, even when an effort is made to share records across organizations, merged records are likely to lead to an accumulation of redundant diagnoses for the same patient if different organizations documented different codes. The issue of multiple different diagnostic codes being applied for the same patient diagnosis still occurs when the various providers work within the same network due to provider oversight. As such, a patient with one diagnosed medical problem may appear as if they have multiple diagnosed medical problems due to the multiple codes. Coding issues are common with ICD-10 Z-codes that correspond to SDoH attributes, as SDoH-informed care is relatively new in healthcare practices. SDoH-informed care is being integrated at increasing rates due to the potential for patient benefit, as well as the expansion of financial and regulatory incentives for integration of SDoH-informed care to existing healthcare practice. Nonetheless, SDoH-informed care is not yet a universally mainstream practice, and adoption of emerging frameworks inevitably comes with growing pains.

In addition to diagnostic codes, similar problems can occur with medication lists. When a patient is prescribed a new medication or discontinues a medication, it is unlikely that every single clinician encountering the patient will be informed of the medication change. Many prescriptions are limited, such as a course of antibiotics, but the prescription will nonetheless remain on a patient's medication history once added until a provider or pharmacist manually removes the prescription. Patients frequently misreport their own medication use. Redundancy may occur if a provider is unfamiliar with the generic name of a medication, fails to recognize the generic name in the patient's record, and documents the brand name (or vice versa). Redundancy may also occur if a dosage is adjusted and the new dose is entered as a new medication without removing the previously recorded dose, such that the medication is now listed twice at two different doses. Data coding quality issues like this example occur frequently and are unlikely to be remedied efficiently. It is not feasible at scale to continuously check for this issue across the volume of patient records, hence, they are often remedied on a case-by-case basis, dependent on whether the issue is detected. Updating patient records in this way is time-consuming and inefficient, and providers rarely have time for careful, in-depth chart review during a patient encounter. Moreover, the charting errors that are the easiest to detect (e.g., redundant diagnoses) are unlikely to be priorities for rectification as they have minimal impact on the provider's role in patient care. A provider can recognize when two diagnoses are redundant and disregard, particularly when the provider has more important items to address in a limited time during a patient encounter. However, the patient EMR/EHR data is used in many other contexts outside of the patient encounter itself.

Many processes that leverage patient EMR/EHR data, such as pharmacy operations, hospital compliance, and insurance claims, rely on partial or total automation in modern settings. If an automated process is not designed to properly identify a particular error, consequences can occur. For example, a patient may be labelled as being a higher risk than appropriate if redundant diagnostic codes for a single medical problem appear in the record as multiple medical problems. If a patient took a 10-day course of antibiotics in months or years prior, but that antibiotic is still listed on a hospital patient's record, the hospital's electronic system may block the pharmacy from filling a medication that is contraindicated for use alongside the antibiotic. Development of patient data analytic tools has been throttled by the unique complexities of real-world patient data.

Data formatting issues impact patient care management independent of whether the process is performed manually (e.g., workers in care management, hospital compliance, insurance underwriting, and so on) or automated/assisted using computational tools. Recognizing errors or inconsistencies in patient data requires strong medical expertise, and the nearly limitless combinatorial space existing for patient data makes the task of programming a computerized tool difficult and unrealistic. Human healthcare expertise is advantageous in that a person may be able to spot problems within patient data that a machine cannot. However, the human mind is also significantly less equipped than a machine to accurately perform large volumes of complex, detail oriented analysis, particularly when the data to be analyzed is derived from multiple sources. Manual review is also plagued by human bias and subjectivity, which is particularly ethically consequential in the healthcare context. Furthermore, manual review is often simply not feasible for most organizations due to staff, time, and financial constraints. As such, automation of patient data management processes is crucial.

It is therefore desirable to develop a patient care management solution that can automate data processing operations, offers robust detection and handling of patient data anomalies, and is sufficiently flexible for a breadth of data input structure and formatting. The technology disclosed meets the aforementioned needs by leveraging AI and machine learning tools that are compatible with unstructured and imperfect patient data. For example, many disclosed methods comprise data pre-processing operations for standardizing data (e.g., ICD-10 Z code transformation), structuring data (e.g., cleaning and labelling tasks). The data pre-processing operations also include augmenting data (e.g., leveraging auxiliary data sources to correct, supplement, and enrich the patient data). As a result, the technology disclosed is enabled to handle diverse types of patient data while mitigating the impact of data quality on machine learning model performance.

Furthermore, the technology disclosed improves upon conventional strategies for handling the complexity and variability of patient data by implementing an ensemble of specialized AI models, thereby achieving a broad-purpose tool with the accuracy offered by specialization. AI-assisted features, according to certain implementations of the technology disclosed, will now be introduced.

### Artificial Intelligence and Machine Learning Models

The disclosed system and methods for care plan generation can include the use of one or more artificial intelligence/machine learning (AI/ML) models. Some implementations may comprise the training of a machine learning model, while others leverage a pre-trained model. In certain implementations, a pre-trained model is fine-tuned or otherwise optimized using a transfer learning approach. The technology disclosed can leverage AI models such as Natural Language Processing (NLP) models or Large Language Models (LLMs). In various implementations, the AI model(s) may be trained to perform single-level or multi-level classification, clustering, extraction, relation extraction, anomaly detection, pattern recognition, and/or generative tasks. In some implementations, the AI model may further comprise an ensemble of machine learning techniques.

A method implementation of the technology disclosed includes invoking at least one callback function that prompts at least one trained AI, running on specialized array processing hardware, to process the patient data and initiate an API request in response to the patient data, in dependence upon a match between the processed patient data and a task that the particular trained AI model is trained to perform. The input data is dynamically adjusted with additional metadata based on auxiliary data supplementation and data pre-processing operations, such as feature engineering. Other implementations of the technology disclosed leverage voice-based interaction, recorded audio or video, or image data in addition to, or in place of, text-based data. For the sake of clarity and conciseness, the example implementations described will primarily refer to text inputs.

Some disclosed methods also include further prompting one or more trained AI models to autonomously engage with other interconnected Internet or cloud-hosted tools and leverage said tools in order to perform tasks responsive to the patient data. In addition to web and cloud services, private libraries and other proprietary collections can be searched to broaden the depth and usefulness of the data associated with a personalized care plan. AI agent services can be trained for generalized learning in order to handle a wide variety of input data, or alternatively, can be trained for a narrow, specialized category of tasks. However, effective deployment of AI models that have been trained at either extreme (e.g., a general knowledge AI agent versus a narrow-purpose AI agent) is difficult to achieve.

Generalization of AI learning to a broader range of knowledge and task automation is often inversely correlated with accuracy of the trained AI model. The problem of maintaining accuracy as the generality of an AI model increases can be attributed to many root causes. One of these root problems is the need for sufficient training data. Real world AI models are typically trained on hundreds of thousands to millions of training examples in order to achieve the accuracy required for quality performance during production. As the overall purpose of the model expands, so does the volume of training data required to achieve sufficient performance (as defined on a task-by-task basis, e.g., an accuracy rate of 90% may be sufficient for an entertainment-purpose chatbot, but insufficient for healthcare use cases). Moreover, imbalances or biases within the training data are detrimental to model accuracy. For example, a model trained on diverse patient data representing a range of health conditions will be more accurate for patients with the most common conditions and less accurate for patients with underrepresented conditions. Many publicly available training data for medical purposes underrepresent racial and gender minorities, as well. Generally speaking, broadening or diversifying a training dataset will increase the likelihood and magnitude of data imbalance.

Another challenge in balancing model breadth and model accuracy is computational cost. In order to perform complex tasks, such as the generation of personalized care plans for a diverse population, the size and complexity of the AI architecture must also increase responsive to the task difficulty. Even in a hypothetical scenario where an adequately large volume of high quality training data is available to train a large AI model, such a scenario is accompanied by extremely high computational power demands. Consequently, the accuracy of broad-purpose AI is limited by the aforementioned prohibitive resource demands associated with expanding model complexity (e.g., data volume, computational resources, time, financial cost). The complexity and variance of medical data, therefore, makes the goal of training one large AI model to process and respond to general population data problematic in light of both model performance and ethical consideration.

In contrast, highly specialized AI operations can be executed for many tasks without the above-mentioned drawbacks of general purpose models. As the intended task or purpose of the model narrows, however, the rigidity of the model increases during production mode. The specialized trained AI model can have very poor accuracy, or fail to address the input entirely, when facing a production input that does not closely align with the training examples. In addition to the type of data input, the format in which the input is presented can also be necessarily restricted for a specialized AI. If an AI model is ill-equipped for flexible input processing, this is a significant hindrance to the utility of the model. Consequently, a specialized model with highly-restrictive input compatibility is also ill-suited for processing patient data because real-world patient data varies greatly.

The technology disclosed presents an innovative solution to the aforementioned problems by providing a hybridized model characterized by the advantages of both generalized and specialized AI, without the disadvantages described above. The technology disclosed leverages an ensemble of specialized AI models, thereby achieving a broad-purpose tool with the accuracy offered by specialization. Many implementations of the technology disclosed involve a plurality of trained AI models corresponding to different tasks, such as a name-entity recognition model for data labelling and classification, a Z-code transformer for SDoH standardization, a large generative model for constructing the personalized care plan, and so on. In some implementations, the AI ensemble is organized such that two or more separate trained AI models corresponding to different tasks operate in tandem. In other implementations, at least two trained AI models corresponding to different tasks operate sequentially such that the output of a preceding model is passed through to a subsequent model as input. Many implementations comprise a combination of tandem and sequential architecture within the AI ensemble.

Input data associated with the patient data is passed through to at least one trained AI model that performs a specialized task responsive to the input, such as autonomously initiating a particular action or callback function, interacting with an API, searching and retrieving information, transforming the input data, or generating an output responsive to the input like a personalized care plan. In certain implementations, a task is identified responsive to a particular input, and the identified task is matched with a best-fit trained AI model within the AI ensemble. Each of the plurality of trained AI models within the ensemble are trained to perform a specific subset of tasks. Hence, the disclosed AI ensemble offers a broad range of functionality without the typical drawbacks of broad-purpose AI systems, and specialized task performance without the typical drawbacks of narrow-purpose AI systems.

Neural network and deep learning architectures utilized by various implementations disclosed include, but are not limited to, Convolutional Neural Networks (CNN), Recurrent Neural Networks (RNN), Deep Neural Networks (DNN), and Transformers. Additional models such as Reformers may be used (e.g., for handling large data sets), and a number of additional machine learning algorithms like Random Forest, Support Vector Machine, clustering and XGBoost either in the place of neural network model(s) or in combination to augment the neural network(s). Furthermore, emerging techniques such as Graph Neural Networks (GNN) and Federated Learning models may be leveraged. In various implementations comprising an NLP model, the NLP model may include Embedding Models that convert text to vectors, thus capturing semantic meanings more effectively. Retrieval-Augmented Generation (RAG) models that combine the retrieval of informational content with generative capabilities to enhance response accuracy are included in certain implementations. Large Language Models (LLMs) such as autoregressive models, which predict subsequent tokens in a sequence, and masked models that predict missing tokens in a sequence, are implemented by some disclosed systems. The suite of NLP technologies disclosed also extends to multimodal contrastive models that learn from diverse data types (e.g., text, image, sound) simultaneously, enhancing the model's ability to process multiple inputs and/or generate multiple outputs.

Many implementations of the technology disclosed include a method for standardizing SDoH data and generating a customizable SDoH care plan. In one disclosed method implementation, clinical notes containing structured or unstructured data (e.g., SDoH data) are automatically translated into a clinical code like an ICD-10 Z-Code. In some implementations, data is translated into multiple clinical codes. Some implementations include leveraging one or more pre-trained AI models, such as a transformer model (e.g., BERT, ROBERTa, DeBERTa, GatorTron, Longformer, etc.). Implementations comprising a pre-trained model may comprise fine-tuning the model using curated SDoH data. Other implementations relate to the training of an AI model, such as a transformer, using a training dataset including curated SDoH data. The said transformer(s) can be trained to perform one or more tasks including, but not limited to, extraction of SDoH features and patterns and/or relation extraction to link one or more ICD-10 Z-Codes to one or more target SDoH classes or features. Various disclosed methods comprise employing prompt engineering to improve accuracy of generated SDoH care plans. In some implementations, the automatic translation of clinical data into clinical codes is applied to billing and reimbursement procedures.

Many implementations of the technology disclosed involve predictive analytics for the identification of a patient's social needs, risks, gaps in care, identifying local resources, or combinations thereof. In various implementations, the technology disclosed includes generating one or more recommendations for service needs based on a patient stated goal or preference (or a plurality of goals and/or preferences). A generated SDoH care plan may comprise a modification of a care plan such as an HL7 C-CDA Care Plan, or similar, to incorporate one or more SDoH-related or targeted, patient-specific precision interventions. A targeted intervention may, for example, comprise behavioral changes, removal of barriers (e.g., housing or transportation needs), changing patient perceptions, improving social support, alleviating fear, prioritizing social needs, identifying relevant local resources, improved management of resources (e.g., capacity planning, user feedback, etc.). Implementations including a generative AI agent and/or prompt engineering can generate various Health Equity Alerts such as Confirmed or Possible Barriers to Care, Health Alerts, Health Risks, missing patient information, and so on. The disclosed generative AI agent can generate one or more prescriptive instructions or recommendations to inform healthcare and clinical decision-making.

While many implementations of the technology disclosed are focused on patient-level data, others are focused on population-level data. For example, many implementations of the disclosed autonomous care navigator SaaS platform include generating one or more patient population insights in addition to (or in place of) generating patient-specific insights. The disclosed autonomous care navigator and/or generative AI agent generate outputs in real-time in many implementations. As previously indicated, certain risks may be associated with the automation of tasks within the healthcare sector due to the prevalence of corner cases, nuances that are unique to patient-specific circumstances, and experiential learning that is difficult to replicate with an AI model.

The technology disclosed harnesses the advantages of machine automation by deploying AI to perform complex data processing operations on data characterized by large volume and high dimensionality with superior accuracy and efficiency than possible when performing the same tasks manually. Furthermore, the problem of providing personalized SDoH care plans in response to patient data requires handling data that is highly inconsistent in terms of data structure, feature representation, and range of values corresponding to specific features. In order to perform said data handling with large-scale objectivity, it is necessary to employ complex logical operations that may be layered, multi-faceted, and/or hierarchical such that it cannot feasibly be performed in the human mind.

The technology disclosed additionally provides a solution to the problem of augmenting data management and automation within the healthcare field without detriment to accuracy via losing the crucial human elements of experience-based learning and ambiguity handling. In order to maintain ethical standards and improve system performance, the technology disclosed provides a graphical user interface (GUI) to (i) improve explainability and transparency of the AI-assisted technology and (ii) facilitate user interactions in order to receive, and respond to, user feedback such as correcting information, updating information, and further customization for a particular patient or population.

### Graphical User Interface

Various implementations further include a GUI to present information to a user (e.g., a provider or care manager) via a dashboard display. The dashboard may present one or more graphical insight representations, e.g., a Health Equity Score, time-series trends of the Health equity Score, identified Focus Areas, Health Equity Impact, Volume of Alerts by Category, Health Disparity Risk, Disparity by Location, Race and Ethnicity Surname Analysis, among others that would be readily recognizable to a user skilled in the art. The SaaS platform may be accessible via a user (e.g., healthcare provider or care team member) portal comprising at least one of a: client computing device, a secured HIPAA-compliant remote application World Wide Web (“Web”) server, an EMR database, cloud-based control service server, said dashboard or GUI, and/or non-transitory computer-readable media. In some implementations, the server may communicate with an EHR or EMR system using at least one API.

The GUI will present a plurality of information to a user associated with the disclosed data operations and models, such as the input data that was processed in order to receive a particular model output (e.g., displaying a list of patient demographic information and diagnoses used by a trained AI model in order to generate a care recommendation). In one use case, the GUI display enables a provider to catch and review potential mistakes or improve the personalized care plan. Some implementations include displaying, via the GUI, a list of flagged discrepancies in the patient data and request, from the provider, any modification or confirmation of each flagged discrepancy. In one example, the patient data includes EMR/EHR data received from two disparate healthcare organizations (such as a hospital network and a primary care private practice that have separate recordkeeping systems). A first medical record associated with the hospital network indicates that the patient takes a particular dose of a blood thinner medication, while a second medical record associated with the primary care provider indicates that the patient takes a different particular dose of the blood thinner. In response to the dosage discrepancy, the GUI can receive a user input from the provider (or other user like a case manager or the patient) confirming an accurate dosage or opting to bypass addressing the dosage discrepancy during the chart review/personalized care plan review process. In some examples, the system will retain the flagged discrepancy in the record and re-prompt the user to review the flagged discrepancy at a later time (e.g., automatically or in response to a user request; at a pre-defined time interval within the interface or a user-defined time interval such as the next time the patient file is accessed, in 10 days, in 30 days, etc.). In some examples, the user can clear the flagged discrepancy from the discrepancy list, with or without addressing the flagged discrepancy via user input.

Other examples of data discrepancies may indicate a plurality of duplicate or highly-similar patient data for the particular patient, such as multiple similar diagnoses that may be redundant, a medication that is listed in one entry under a generic name and another entry under a brand name, or two distinct data sources relating to respective patient encounters that may be referring to one patient encounter. Similarity can be measured using one or more of an auxiliary data source (e.g., a key-value dictionary or relational database categorizing medications or diagnoses), a pre-defined threshold for overlapping language or quantity of shared keywords, or computational operations like distance within a vector/tensor space. Data discrepancies may also include a reported medication that conflicts with a reported allergy. Data discrepancies may also include flagged care events for the patient, such as a documented referral to a specialist and no documentation on record that the patient followed up on the referral. A provider may be prompted to remind the patient to schedule an appointment, or follow up with a patient after a procedure, for example. Furthermore, the provider can review AI-assisted operations to assess the AI-assisted operations for ethical or accuracy concerns and provide user feedback to correct or augment the data. In some implementations, the provider can initiate a report associated with the AI-assisted operation for further review to improve upon model accuracy. In other implementations, user feedback is collected and incorporated into training data to improve the accuracy of future versions of the disclosed system.

Other forms of user feedback that can be received by the GUI can include, for example, progress notes documenting completion of the patient care plan, updated patient data, patient-reported satisfaction or quality of life, and so on. In some implementations, the patient can prompt the GUI to provide further explanation or educational information related to the patient's personalized care plan. In other implementations, the patient can provide feedback on the ease of understanding, accessing, or using a recommended treatment or intervention. In response to patient feedback, the GUI may pass this information to other components of the disclosed system via an API layer in order to autonomously initiate one or more of a notification to a provider, an update to the patient data, or usage of the patient feedback data within training data for a machine learning model.

In many implementations, the GUI allows a user to drill down further on a displayed graphical element or data to provide further information. For example, the GUI may provide an overview interface with key statistics and recommended care interventions for a patient without overly cluttering the interface with excess data to improve accessibility. The user can drill down further on a particular statistic or care intervention to see more information about how a computation was performed or why a recommendation is being made. The disclosed system may also perform additional analyses upon request from the user via the GUI, such as a trend analysis, a progress report, or population-level statistics based on a plurality of patients sharing at least one characteristic of interest.

By presenting a range of patient data, analyses, and care recommendations for a patient in a user-friendly, accessible format and facilitating user interaction, the disclosed GUI enables more transparent and explainable healthcare AI. This results in a more ethical implementation of AI because it allows providers to better understand the generated output, quickly identify areas of concern, and address areas of concern to mitigate risk of negative patient impact. The information is presented in a user-friendly way, allowing for users with non-technical backgrounds to utilize AI-based tools at a much deeper level that is often only accessible to those with highly technical computational backgrounds. Moreover, user interaction with the system can be stored and further leveraged to improve overall functionality of the system by correcting existing quality issues. User interaction features within the GUI lower the educational barrier for a provider, patient, or other system user to understand the personalized care plan, patient health, and why certain assessments or recommendations are being made. User feedback can be incorporated within feedback loops to improve upon model accuracy at a fine-grained resolution (i.e., improving the personalized care plan for a particular patient) or a coarse-grained resolution (i.e., future model training to improve model performance for generating any personalized care plan).

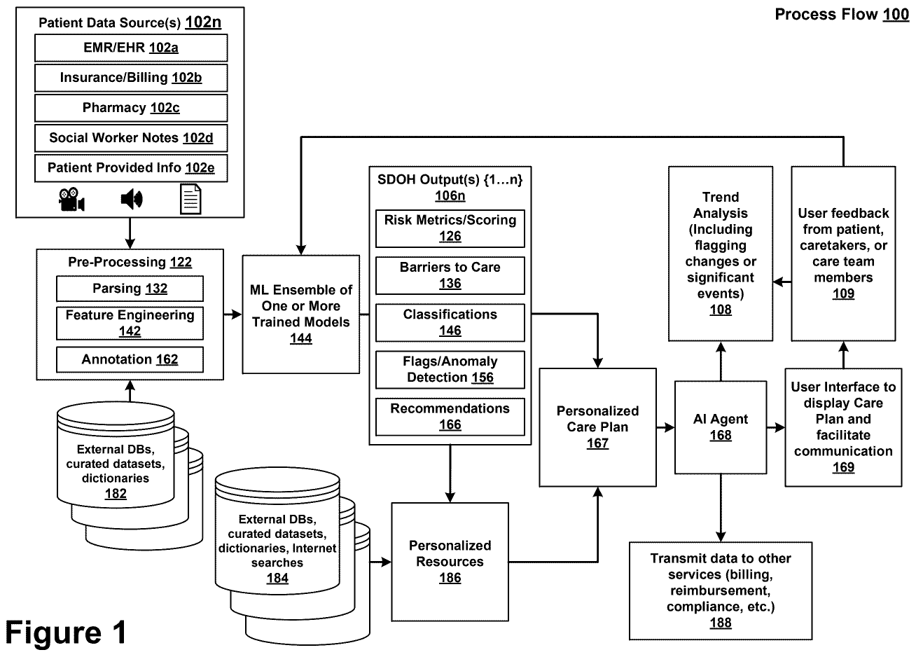

The discussion now turns to additional particular implementations of the technology disclosed with reference to FIGS. 1-13.

### System Overview

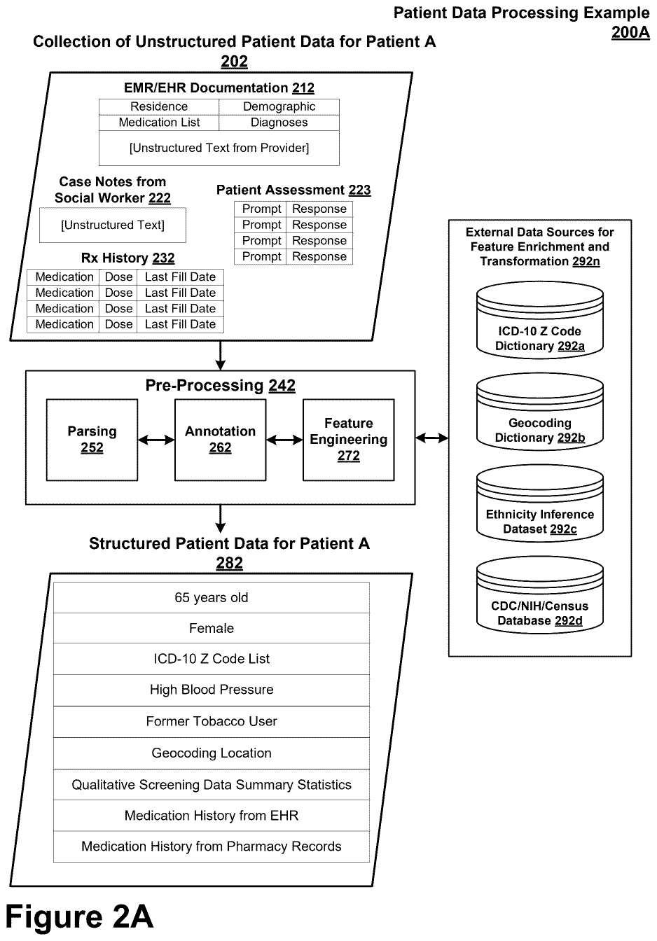

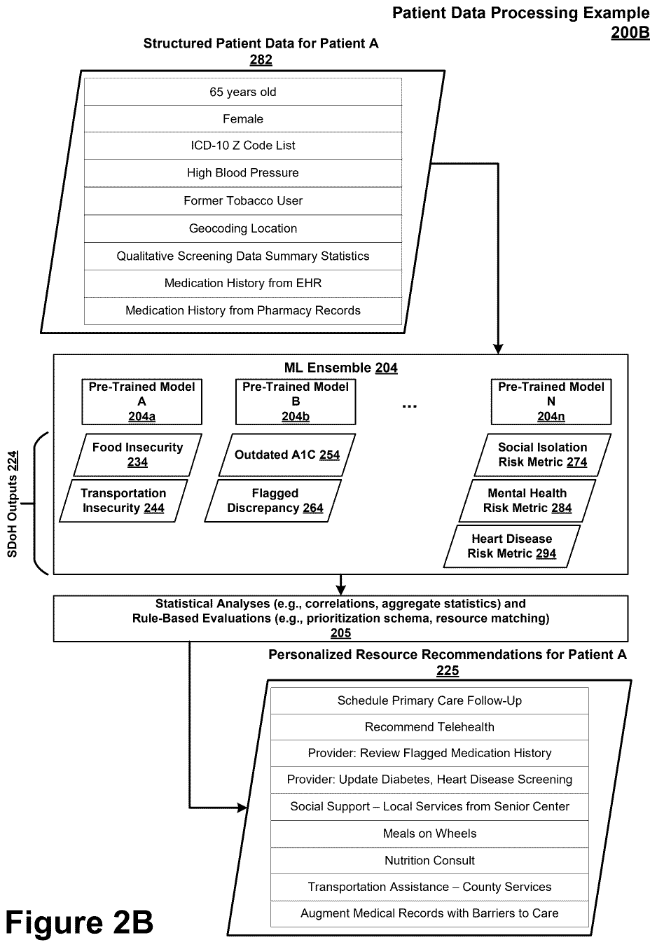

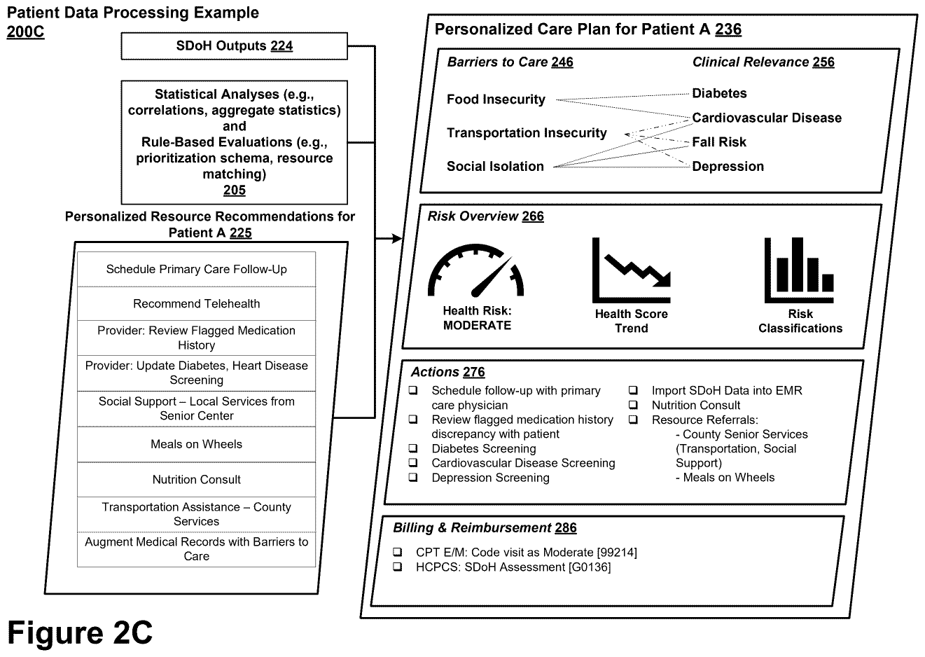

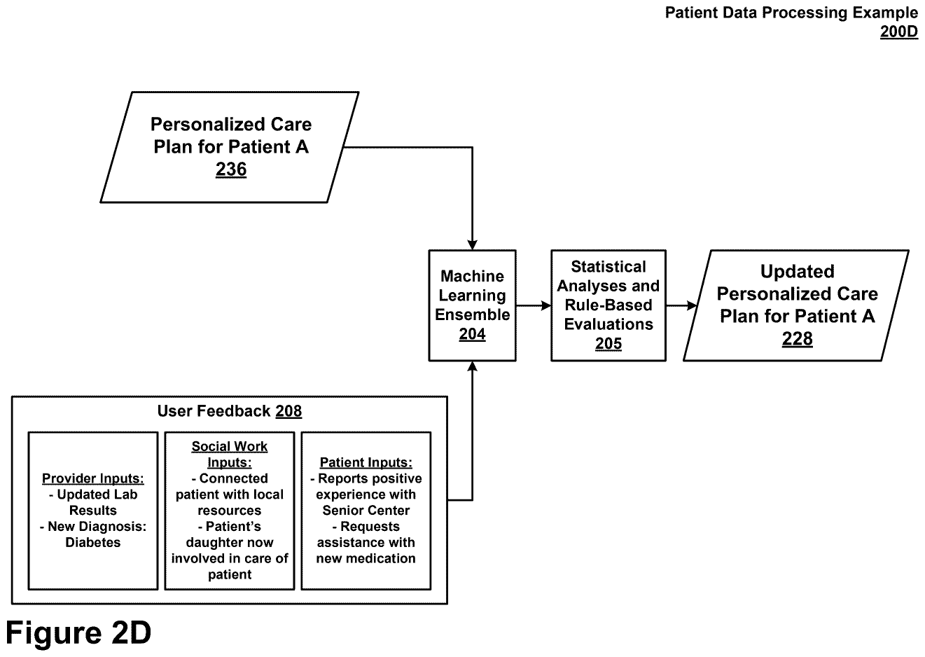

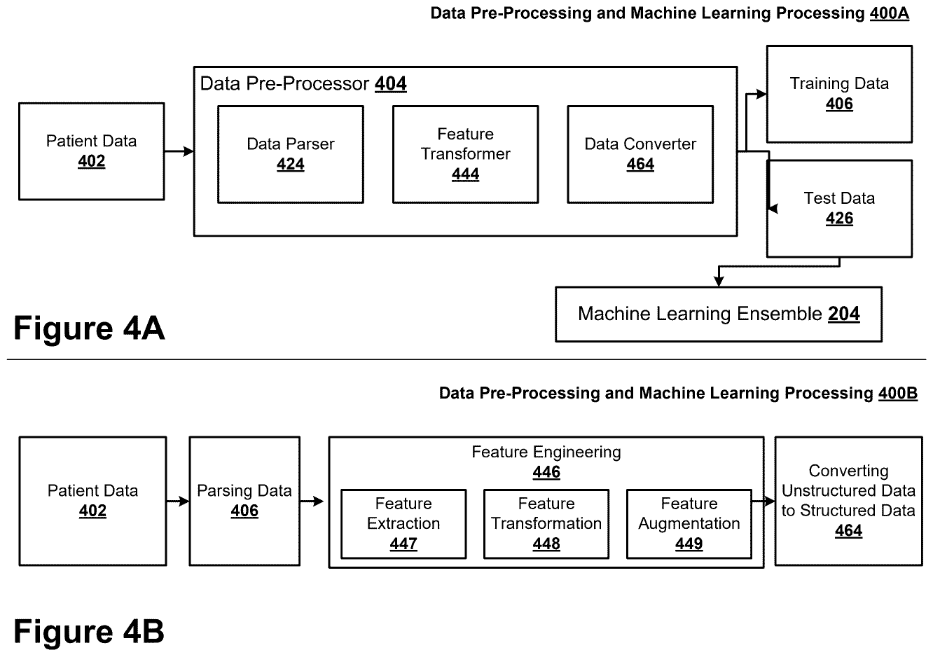

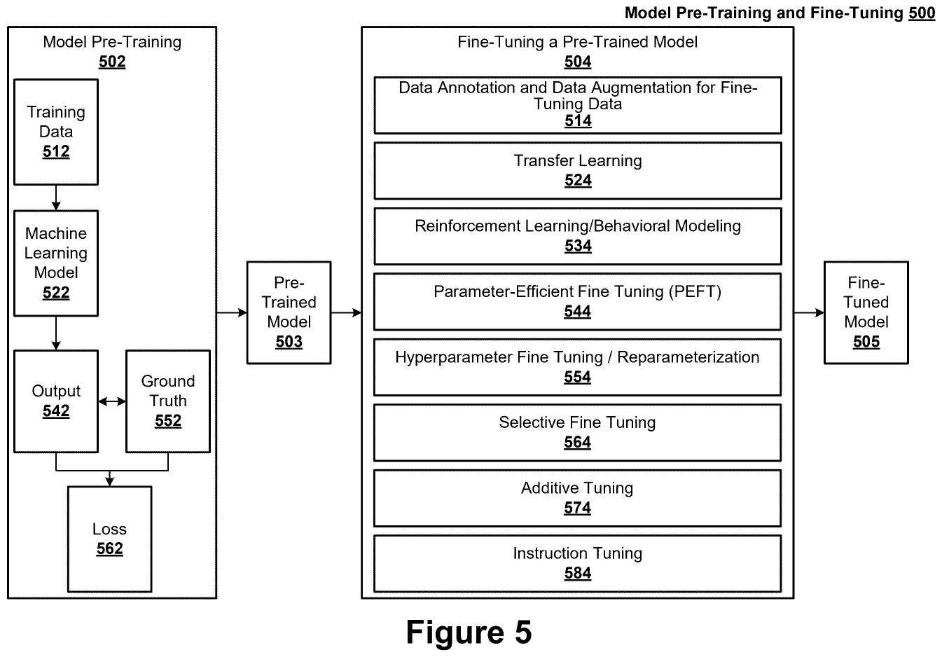

FIG. 1 is a schematic diagram 100 illustrating generation of an end-to-end social determinants of health (SDOH) management system. Schematic diagram 100 includes a plurality of patient data sources 102n. Examples of disparate patient data sources can include EMR/EHR 102a, insurance and billing data 102b, pharmacy records 102c, social worker notes 102d, patient provided information 102e (e.g., patient messages in a patient/provider communication system or patient-reported survey responses), and other sources of patient data not shown. The patient data may include sources of video and audio as well as textual data. The data obtained from patient data sources 102n undergoes a series of pre-processing operations 122, including data parsing 132, feature engineering 142, and annotation 162 (described in further detail with reference to FIGS. 4A, 4B, and 5). The data pre-processor can access one or more auxiliary data sources including external databases, curated datasets, and dictionaries (e.g., key-value dictionaries and coding dictionaries) 182 to augment the patient data. The data pre-processing operations 122 further include converting the unstructured patient data into structured patient data including any engineered or augmented features and/or data labels. The structured patient data is processed by a machine learning ensemble 144, including one or more trained machine learning models. The machine learning ensemble 144 processes the structured patient data in order to generate one or more SDoH output(s) 106n. SDoH outputs 106n may include, for example, risk metrics and risk scores 126, detected barriers to care 136, patient classifications 146, anomaly detection and anomaly flagging 156, and/or patient care recommendations 166. The SDoH outputs 106n are used to generate one or more prescriptive personalized resources. Generation of the personalized resources 186 can be assisted by auxiliary data sources such as including external databases, curated datasets, and dictionaries 184 (which may be non-overlapping with data sources 182 or there may be one or more overlapping data sources between 182, 184). A personalized care plan 167 can be generated based on a combination of the SDoH outputs 106n, personalized resources 186, and statistical post-processing thereof. The personalized care plan 167 is presented to a user (e.g., a patient, provider, or care manager) via an AI agent 168. The AI agent 168 enables the user to interact with the personalized care plan, via a user interface 169, including reviewing information within the personalized care plan, modifying the personalized care plan, and/or providing progress updates for the personalized care plan. This user feedback from the patients, caregivers, providers, etc. 109 is used to inform trend analyses 108 (e.g., anomaly detection and progress tracking) and used within a feedback loop integrated into the machine learning ensemble 144 to continuously update the personalized care plan 167 with up-to-date and correct information. FIGS. 2A-2D provide an in-depth example of this process with respect to an illustrative patient, in accordance with some implementations of the technology disclosed.

FIG. 2A is a flow diagram 200A illustrating an example of generating a personalized care plan from patient data. A collection of unstructured patient data for a Patient A 202 is curated from a plurality of disparate data sources. Some data sources, such as the EMR/EHR documentation 212, can include a mixture of structured data (fields for attributes including patient residence, demographic data, medications, and diagnoses) and unstructured data (provider notes). Some data sources, such as case notes from a case manager or social worker 222, may include unstructured text data without any structured data dedicated to particular attributes. Other data sources, such as a patient assessment 223 or a prescription history 232, may include entirely structured data. However, even though some data sources may include at least a portion of structured data, it is unlikely that any two disparate sources of patient data will have the same structured data or formatting. For example, as illustrated by unstructured patient data 202, all four example data sources (EMR/EHR documentation 212, case notes 222, patient assessment 223, and medication history 232) have unique structure differing from the other data sources within unstructured patient data 202. Furthermore, the EMR/EHR documentation 212 and medication history 232 both include medication data, but this data can be formatted entirely differently in each respective source, making it difficult for data processing operations to properly parse and interpret the medication history across the plurality of data sources.

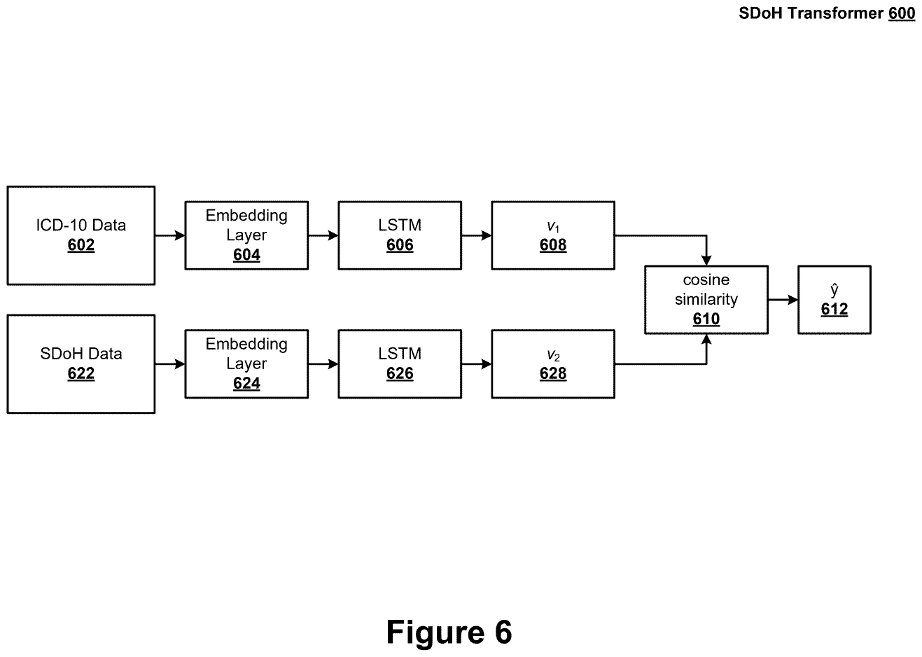

In order to address the issues associated with unstructured (or mixed structure) patient data, a data pre-processor performs a plurality of data pre-processing operations 242 in order to convert unstructured patient data 202 into structured patient data 282. Pre-processing operations 242 include parsing 252, annotation, 262, and feature engineering 272. The parsing operations 252 can include leveraging natural language processing techniques to parse unstructured text, such as provider or case worker notes, in order to identify relevant keywords so that this information can be re-structured into a cleaned, formatted data structure. The annotation operations 262 can include labelling the data to enrich the patient data and provide key context associated with the data. Feature engineering operations 272 can include feature transformation, feature augmentation, and/or feature selection. Feature transformation can include, for example, SDoH Z-Code transformation (discussed further with reference to FIG. 6) in order to standardize SDoH data. Feature augmentation can include introduction of novel features, obtained from analyses of existing features to extract more information, or obtained from auxiliary external data sources for feature enrichment and transformation 292n. These external data sources may include, for example, an ICD-10 code dictionary 292a, a geocoding dictionary 292b, an ethnicity inference dataset 292c, and/or a dataset 292d curated from sources like the CDC, NIH, or government census data. In one example of feature enrichment, the geocoding dictionary 292b can be leveraged to augment patient data 202 with additional information associated with the ZIP code in which Patient A resides (e.g., a food desert). In another example, the ethnicity inference dataset 292c can be used to supplement missing race/ethnicity data to flag potential risks correlated with ethnicity.

Pre-processing operations 242 generate structured patient data for Patient A 282. The structured patient data 282 includes a structured, standardized version of the unstructured data 202 with augmented feature representations. This structured data 282 is a higher quality input for later analyses, such as machine learning processing. FIG. 2B continues the example of FIG. 2A with a flow diagram 200B illustrating machine learning processing of structured data 282.

Diagram 200B includes a machine learning ensemble 204 comprising one or more pre-trained machine learning models, e.g., Pre-Trained Models A 204a, B 204b, and N 204n. Each respective pre-trained model has been trained (and in many cases, fine-tuned) for a specific subset of tasks. Examples of pre-trained machine learning models are provided throughout the discussion herein, such as a risk assessment model or a Name-Entity Recognition Model. The respective machine learning models 204a, 204b, and 204n each produce a number of corresponding SDoH outputs 224. For example, pre-trained model A 204a processes patient data 282 as input in order to generate, as output, barriers of care for the patient like food insecurity 234 or transportation insecurity 244. Pre-trained model B 204b is an anomaly detection model that processes patient data 282 in order to identify anomalous data, such as an outdated A1C value 254 or a data discrepancy 254, that should be addressed in order to improve the overall quality of care for Patient A. Pre-trained model N 204n is a risk assessment model that processes patient data 282 in order to generate a plurality of risk metrics, such as a social isolation risk metric 274, a mental health risk metric 284, and a heart disease risk metric 294.

One or more of the SDoH outputs 224 undergoes further statistical analyses and rule-based evaluations 205. For example, correlative data and significance testing can provide additional information about how to properly address the identified SDoH data for Patient A. Furthermore, prioritization schema and resource matching evaluations enable a user to act on the model outputs of ensemble 204 by identifying priorities for the patient's care and appropriate interventions responsive to said priorities.

The accumulation of SDoH outputs 224 and downstream analyses thereof 205 is then leveraged by an AI agent to generate a personalized resource recommendation for Patient A 225. The personalized resource recommendation can include, for example, recommendations to schedule a primary care follow-up visit, set up telehealth services responsive to a detected transportation access barrier, initiate social support services in response to a social isolation risk metric value above a pre-defined threshold, and so on. Next, FIG. 2C illustrates a personalized care plan for Patient A 236 based on the SDoH outputs 224, downstream analyses 205, and personalized resource recommendations 225 in diagram 200C.

Personalized Care Plan 236 provides a transparent, accessible representation of the AI-generated outputs that helps both patients and their care teams understand the SDoH data, interact with the SDoH data, and act on the SDoH data. For example, Personalized Care Plan 236 shows a graphical representation of the detected barriers to care 246 for Patient A (e.g., food insecurity, transportation insecurity, and social isolation) and the clinical relevance 256 of said barriers to care (e.g., diabetes, cardiovascular disease, fall risk, and depression). A risk overview 266 graphical element displays risk data in an easily understandable format. For example, the plurality of generated risk scores by the machine learning ensemble 204 can be aggregated into an overall Health Risk score. The health risk score can be classified into high risk, moderate risk, or low risk, based on pre-defined score range boundaries. Alternate implementations that variously aggregate risk scores or representations of risk metric data in qualitative or quantitative formats will be readily apparent to a user skilled in the art. As risk data is obtained over time, trend analysis can be performed to indicate if health risks are being reduced or compounded for the patient over time. Risks may also be classified into particular categories, such as social isolation risks or cardiovascular health risks. An action list 276 is constructed for the provider based on the AI-assisted analysis of patient data, such as resource referrals, resolving EMR/EHR discrepancies, or further health screenings (e.g., diabetes testing). Furthermore, billing and reimbursement 286 can also be automated and summarized within the Personalized Care Plan 236.

FIG. 2D illustrates a flow diagram 200D for updating the Personalized Care Plan 236 in dependence upon user input 208. User input, received via a care navigator GUI, can be obtained from the provider, social worker, patient, etc. Examples of provider feedback may include updated lab results or new diagnoses. Examples of social work feedback may include resource interventions or lifestyle updates. Examples of patient feedback may include satisfaction survey responses or requests for assistance. The Personalized Care Plan 236 and user feedback 208 can be processed via the machine learning ensemble 204 and downstream analyses 205 in a feedback loop to generate an Updated Personalized Care Plan 228.

The discussion now turns to an autonomous care navigator, an AI agent-assisted system for implementing the workflows described with reference to FIGS. 1 and 2A-2D.

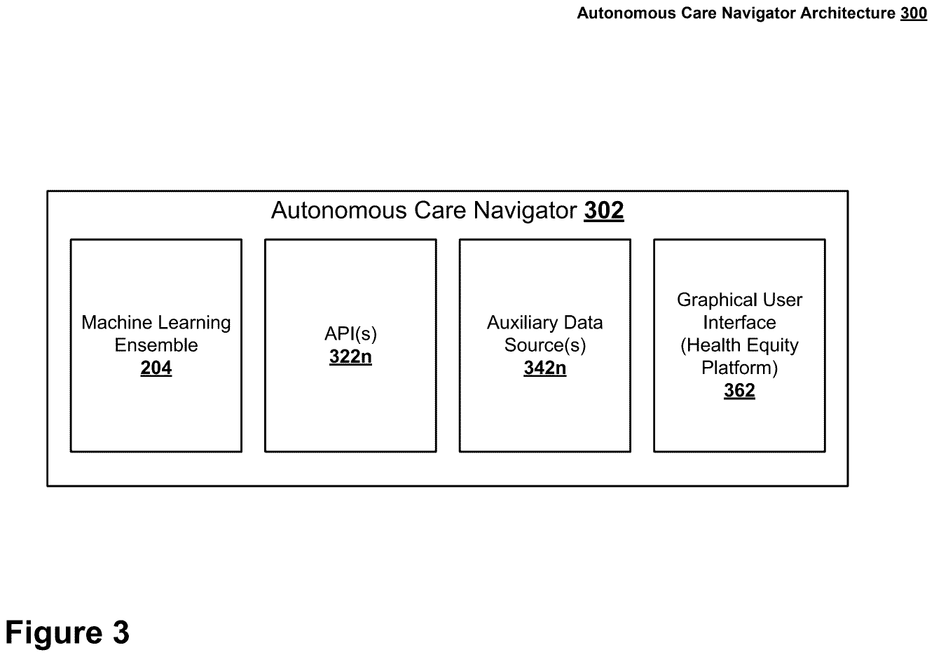

FIG. 3 is an architectural diagram 300 of an autonomous care navigator 302 through which one or more aspects of the technology disclosed may be implemented. The autonomous care navigator 302 further comprises a health equity platform GUI 362 and a machine learning ensemble 204. In many implementations, machine learning ensemble 204 comprises an AI agent (e.g., a generative AI) or interacts with the AI agent, such as AI agent 168 of FIG. 1 (not shown in FIG. 3). The AI agent 168 can communicate or otherwise operatively engage with the health equity platform 362 via one or more APIs 322n. The autonomous care navigator 302 may further integrate at least one auxiliary data source 342n, such as an external database. Auxiliary data sources 342n are accessible to the health equity platform 362 and AI agent 168. A particular auxiliary data source 342n can be associated with an EHR system, EMR system, CRM system, and so on. The AI agent 168 of autonomous care navigator 302 can be trained to perform tasks such as identification of one or more barriers to care, prioritization of one or more barriers to care, automatic capture of one or more Z-code, standardization of one or more Z-code, automatic capture of at least one reimbursement-related charge, identification of one or more social risk, prioritization of one or more social need for a patient, identification of resources in dependence upon prioritized needs, or combinations thereof. Herein, many implementations of the technology disclosed are described with reference to ICD-10 Z-Codes. However, it is to be understood that any alternative clinical coding system may be used in place of ICD-10 Z-Codes and the implementations described are merely limited to Z-Codes for clarity and conciseness.

The AI agent 168 is trained to generate a patient-specific, longitudinally adaptable SDoH care plan that may be utilized to augment productivity, reduce patient health disparities, and standardize care. It is to be understood herein that the generated SDoH care plans are described with varying language throughout (e.g., “personalized,” “customizable,” “patient-specific,” etc.) but unless explicitly stated otherwise, terms similar to “personalizable,” “customizable,” etc. are used synonymously. The generation of a patient-specific, longitudinally adaptable SDoH care plan is described further above with reference to FIGS. 2A-2D.

FIG. 4A is an architectural diagram 400A for a data pre-processor 404, according to some implementations of the technology disclosed. The architecture of diagram 400A comprises patient data 402, a data pre-processor 404, and machine learning ensemble 204. In various implementations of the disclosed system, the data pre-processor 202 further comprises one or more of a data parser 424, a feature transformer 444, and a data converter 464. Once the patient data 402 has been parsed for key information (e.g., extracting a health risk term from a patient note) and the features have been extracted, transformed, and augmented, the data converter 464 converts the patient data 402 from an unstructured format into a structured format. The structured data can be used as test data 426 for the machine learning ensemble 204, or stored as a training dataset 406 for future training of a machine learning model. In one implementation, the machine learning model is an AI/ML model that may perform supervised, semi-supervised, or unsupervised learning tasks. In one implementation, a data annotation logic (not shown) performs annotation of data prior to the annotated data being split into training dataset 406 and test dataset 426. One or more outputs of the data pre-processor 404 can be provided as input to an NLP model. The NLP model processes the data received from data pre-processor 404 as input in order to generate, as output, one or more Named Entities 222 (e.g., a particular SDoH attribute). In some implementations the NLP model is a Named Entity Recognition (NER) model, while in others, an NLP model operates cooperatively with an associated NER model (or a plurality of NER models) in order to generate the Named Entities. A Named Entity 222 may be, for example, a SDoH data type such as marital status or transportation access, a patient symptom, a disease or disease state, and so on.

In many implementations, the performance of one or more AI/ML models within ensemble 204 is evaluated by an evaluator logic (not shown). The evaluator logic further comprises a quantitative evaluation logic and/or a qualitative evaluation logic, which respectively generate quantitative performance evaluation metrics and qualitative performance evaluation metrics. Performance metrics used in the evaluation of an AI/ML model can include a loss function, accuracy rate, F-score, and others readily apparent to a user skilled in the art.

FIG. 4B shows an example workflow 400B for pre-processing patient data 402, according to some implementations of the technology disclosed. The care plan generation workflow depicted in 200B comprises a data parsing operation 406, feature engineering 446 including feature extraction 447, feature transformation 448, and feature augmentation, and data conversion from unstructured data to structured data in operation 464. Input data can include, for example, patient data such as one or more data sources may comprise a data type or element (e.g. USCDI), including but not limited to, patient demographic, geolocation, Zip Code, patient address, encounter identifier, medication status and history, International Classification of Diseases (ICD) code(s), Current Procedural Terminology (CPT) code(s), Healthcare Common Procedure Coding System (HCPCS) code(s), National Drug Code (NDC) code(s), Logical Observation Identifiers Names and Codes (LOINC), Systematized Nomenclature of Medicine Clinical Terms SNOMED, Screening Question Code, Screen Procedure Code, Assessment/Diagnosis Code, SDH Code, clinical notes, clinical test results, encounter metadata, goals of care, health concerns, laboratory test data, procedure history, vital signs, documented health problems, etc., located within an internal or external database.

In some implementations, a data preparation operation (not shown) includes additional data cleaning of patient data 402 such as clinical notes or case reports, prior to the data parsing operation 406 at which point the data is transformed into a data frame format. Data preparation can include a data indexing operation, in which the transformed data frames are indexed, thereby creating a SDoH term database. The transformed and indexed data can additionally undergo further annotation and data labelling in a labelling operation in order to generate a labelled training dataset. Labels may be generated automatically via an algorithmic process or manually by subject matter expert (SME) annotation. The generated labelled training data can be used to trade one or more AI models. The AI model(s) may be, for example, an NLP model, a Large Language Model (LLM), a transformer, a generative AI, a NER model, and so on. In implementations comprising an ensemble of multiple AI architectures, the respective AI models may be trained in isolation or holistically. The data may be labelled using schemes such as IO, IOB2, or IOBES annotation. In some implementations, the AI model is pre-trained and undergoes fine-tuning or transfer learning (described further with reference to FIG. 5).

Many implementations comprise a NER model that is trained to perform Named Entity identification, classification, or a combination of both in order to generate one or more Named Entities. Named Entity identification further comprises the NER model retrieving one or more entity tokens from the input data. Named Entity classification further comprises assigning one or more classes to each of the identified entities. In some implementations, the Named Entities reflect SDoH data elements such as those identified within the United States Core Data for Interoperability (USCDI).

Many implementations of the technology further include a relation extraction operation (not depicted in FIGS. 1-13), in which a relation extraction engine processes the Named Entities as input in order to generate, as output, data associated with SDoH relations. Some implementations further include a care plan generation operation in which one or more determined SDoH relationships are processed to generate an SDoH care plan. The generated SDoH care plan may include, for example, output data such as a health concern or health risk, a prescriptive patient goal, a barrier to care, a prescriptive intervention, a predictive outcome, preference, order, or coordination.

One or more pipelines or architectures of the disclosed autonomous care navigator system may perform one or more of the aforementioned operations described with reference to workflow 400B for the generation of a patient-specific or customizable SDoH care plan from one or more curated, annotated corpus of SDoH, or ontology. An ontology may include formal representations of an SDoH specific domain to facilitate semantic interoperability across systems with formal definitions of concepts and their relationships. In various implementations, ontologies useful for SDoH plan development may include but are not limited to, Ontology of Medically Related Social Entities (OMRSE), focusing on health-related social roles, the Semantic Mining of Activity, Social, and Health (SMASH) data system ontology, focusing on the interrelations of health, social activities, and daily physical activities, contextual-level ontologies such as the Environment Ontology (ENVO), the Human Health Exposure Analysis Resource (HHEAR) ontology, the Child Health Exposure Analysis Resource (CHEAR) ontology, and the Environment Conditions, Treatments, and Exposures Ontology (ECTO), among others.

Many implementations of the technology disclosed leverage fine-tuning of pre-trained machine learning models. In a model training workflow 502, training data 512 is used to train a parameterized machine learning model 522. The training output 542 is compared to ground truth labels 552 for the data in order to obtain a loss 562, which is minimized during training. This pre-trained model 503 can be, for example, a large language model trained to perform NLP tasks. The pre-trained model 503 can be further fine-tuned to improve performance of pre-trained model 503 for healthcare tasks. Fine-tuning operations 504 can include data annotation and data augmentation for fine-tuning 514 (e.g., fine-tuning datasets including SDoH data with ground truth labels). Fine-tuning operations can also include transfer learning 524 or reinforcement learning 534. In some implementations, fine-tuning operations 504 include parameter-efficient fine tuning (PEFT) 544 to selectively fine-tune certain parameters without risking catastrophic forgetting of information learned during training by modifying key learned parameters. Other hyperparameter fine-tuning/reparameterization operations 554 or selective fine-tuning 564 may also be used other than PEFT 544. In some implementations, additive tuning operations 574 introduce new parameters to the pre-trained model 503. In many implementations, instruction tuning 584 is used to constrain the outputs generated by pre-trained model 503. The fine-tuning operations 504 result in a fine-tuned model 505.

Many implementations of the technology disclosed leverage an AI agent for generation of an SDoH care plan. The AI agent, such as the AI agent 168 of FIG. 1, generates one or more care plans tailored to individual health conditions, local resources, and/or patient demographics. In many implementations, a generative AI agent implements a plurality of LLMs for intricate personalization and capabilities for addressing bias in predictive outputs. LLMs are trained to process tokenized text. Tokenization may comprise the parsing of text into non-decomposing units called tokens. Tokens can be characters, sub-words, symbols, or words, depending on the size and type of the model. Tokenization techniques that can be utilized by the technology disclosed include, for example, WordPiece, Byte Pair Encoding, or Unigram LM.

In one implementation leveraging a transformer model, an encoder encodes the input sequences to variable length context vectors, which are then passed to a decoder to maximize a joint objective of minimizing the gap between predicted token labels and the actual target token labels. Non-limiting examples of encoder transformers are DistilBERT, ALBERT, BERT, ROBERTa, ELECTRA, and so on. Non-limiting examples of encoder-decoder transformers are mBART, T5, BART, and so on. In some implementations, the disclosed LLM comprises a decoder without an encoder. Non-limiting examples of decoder transformers are GPT, GPT-2, GPT-3.5, GPT-4, CTRL, and so on. Some implementations leverage transformer architectures including multiple attention heads, aiding in the understanding of context and relationships between SDoH related text, words, concepts, ontologies and one or more patient health conditions or outcomes. As referenced herein, attention mechanisms can include non-limiting selection attention, self-attention, cross attentions, full attention, sparse attention, flash attention, and others readily recognizable to a user skilled in the art.

Many implementations of the technology disclosed comprise a plurality of LLM operations, including pre-training through deployment of prompt/utilization. The training pipeline for the LLM may comprise data processing operations performed on a large corpora of text data (e.g. clinical notes, USCDI, etc.) to be used for model training. Techniques for pre-processing include quality filtering, data de-duplication, and privacy reduction. Preliminary training operation(s) include the training of a model on a large-scale, pre-processed training dataset, and more specifically, training the model to perform language modeling objectives such as full language, prefix language, masked language, or unified language modeling. In one example implementation, the model is trained using self-supervised learning on a large corpus to predict subsequent tokens given a preceding input.

In a fine-tuning operation, the pre-trained model may be optimized using a smaller, labeled data set curated for task specificity, i.e., transfer learning. In order to enable the model to effectively respond to user queries, the pre-trained model is fine-tuned through an instruction tuning operation using instruction-formatted data (i.e., instruction and an input-output pair). For example, the instructions can include multi-task data in natural language provided for guiding the model in response generation according to a prompt in combination with the input. Manual auditing by a human reviewer can be leveraged to monitor the model development for false, biased, or potentially harmful results (e.g., explicitly or implicitly discriminatory outputs). The model can be further trained using one or more reinforcement learning techniques, including a reinforcement learning with human feedback (RLHF) approach. Reinforcement learning techniques may be further augmented with behavioral learning strategies such as reward modeling. In a reinforcement learning framework, an AI model is trained to successfully interact with its environment (usually a simulated environment), as defined by task-specific goals. While the AI performs actions within its environment, an iterative feedback loop of a reward function (positive reinforcement) and/or a loss function (negative reinforcement) guides the training of a model, such as an AI agent comprising an LLM to better accomplish the pre-defined goals provided to the AI agent. The algorithm processes a current state in order to prescribe an action responsive to the state. Depending on how well the prescribed action aligns with the pre-defined goals (e.g., as defined by a behavioral policy probability distribution), a reward is computed, and the model training process continues iteratively in order to maximize the reward function output.

In one example implementation, a pre-trained reward model ranks the LLM-generated responses as preferred or non-preferred outputs which are then used to align the model with an optimization policy (e.g., proximal policy optimization). The policy optimization process is executed iteratively until convergence or a pre-defined threshold. In other implementations, the reinforcement learning techniques may be configured for model adaptation based on dynamic user feedback. The feedback, collected from providers, researchers, case managers, patients, etc., can be collected directly from the user or indirectly via tracking modifications in the care plan.

In another implementation, the reinforcement learning and/or reward modeling processes may incorporate deep Q-learning with the goal of adapting and refining the model based on dynamic user and patient feedback. Q-learning is a discrete domain (e.g., up, down, left, right), value-based (e.g., DQN), off-policy, model free (i.e., making no assumptions of its operating environment) control algorithm. Q-learning may be useful for finding optimal strategies within an environment for which neither the transition function nor the probability distribution of state variables is known.

Some implementations of the technology disclosed further include a prompting operation, in which a query is provided to a trained, fine-tuned (i.e., adapted to a particular task) LLM in order to prompt the LLM to generate an output. Prompting frameworks include non-limiting zero-shot prompting, in-context learning, reasoning, “including but not limited to,” Chain-of-Thought (CoT), Self-Consistency, Tree-of-Thought, single-turn instructions, multi-turn instructions, and so on. The prompting operation can be used to generate a comprehensive SDoH care plan.

In some implementations, the model is fine-tuned indirectly by augmenting and fine-tuning the input data to standardize the data format, which consequently improves model outputs. Many implementations of the technology disclosed further include automatic translation operations for the standardization of SDoH data.

### Social Determinants of Health Data Standardization

FIG. 6 is an architectural diagram of an ICD-10 Z-Code Transformer 600 for SDoH data standardization, according to some implementations of the technology disclosed. Transformer 600 comprises one or more Siamese networks that are trained to perform learning and transforming one or more ICD-10 Z-Codes into SDoH data, concepts, or descriptions. Other architectures are used in alternate implementations, such as an autoencoder. The one or more deep learning networks associated with transformer 600 employ techniques including one shot learning and negative cost computation for precision data SDoH data mapping. One or more ICD-10 Z-Code and SDoH descriptions are transformed into their respective numerical word representations, i.e., embedding of word vectors. A word embedding is a language modeling and feature learning technique used in NLP in which words are mapped to vectors of real numbers with varying dimensions. These word vectors are positioned in a vector space such that words that share similar contexts in the corpus are situated close to one another in the space. In various implementations, input data including at least one ICD-10 Z-code 602 is fed into a first embedding layer 604. The output of the first embedding layer 604 is fed into a first long short-term memory (LSTM) network 606, or alternatively, another type of recurrent neural network (RNN). The output of the first LSTM network 606 comprises one or more v1 vectors 608. Some implementations further comprise a parallel process in which additional SDoH data 622 is inputted into a second embedding layer 624. The output of the second embedding layer 624 is fed into a second LSTM network 626 or other deep learning architecture. The output of the second LSTM network 626 may comprise one or more v2 vectors 628. In various implementations, output v1 vectors 608 and v2 vectors 628 are inputted into a cosine similarity module 610 which generates an output ŷ612 derived by calculating cosine values of term vectors for the given vectors v1 608, v2 628. In various implementations, an objective cost function is used comprising the mean of the following two cost functions:

$$Cost 1 = max ( - cos ( A , P ) + mean\_neg ) + α , 0 )$$

$$Cost 2 = max ( - cos ( A , P ) + closest\_neg ) + α , 0 )$$

$$Cost\_Full = Cost 1 + Cost 2$$

In an alternative implementation, similarity module 610 may comprise the use of Manhattan or Euclidean distance to identify one or more sentence similarities. In yet another alternative implementation, the objective cost function may comprise a mean-squared error loss.

### Implementation of Artificial Intelligence/Machine Learning Models

Many implementations of the technology disclosed include a transformer model, such as transformer 600, that is pre-trained using a large language dataset to generate SDoH output predictions. The predictive capabilities of a pre-trained model may then be transferred in an operation via transfer learning techniques including feature engineering or fine-tuning using a domain-specific corpus or data, e.g., ICD-10 Z-code or SDoH data.

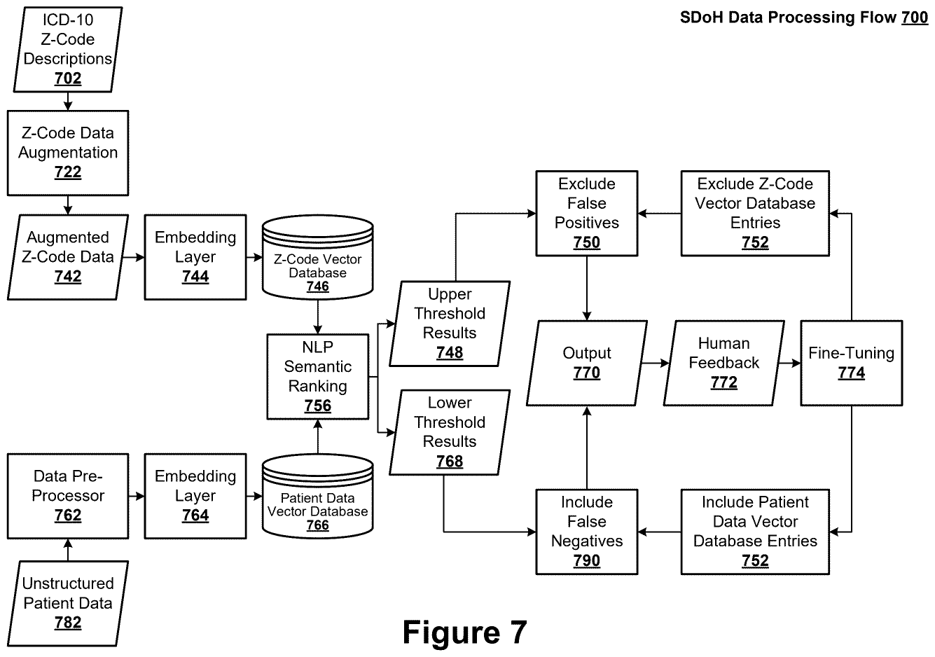

FIG. 7 is a schematic diagram 700 depicting an example algorithm for extracting SDoH-related terminology from patient data. The depicted SDoH terminology extractor algorithm comprises a Z-code augmentation process 722 having one or more inputs of ICD-10 Z-Code descriptions 702. The SDoH terminology comprises one or more phrases with a clinician or physician's clinical notes of a patient's encounter. The Z-code augmentation process 722 generates one or more augmented Z-code data outputs 742 which subsequently serve as, or provide, data input into a first embedding layer 744. The first embedding layer 744 then generates one or more vectors representing a translation of the Z-codes that are stored in a Z-Code Vector database 746. Concurrently, the extractor algorithm performs the pre-processing 762 of one or more sources of patient data 782 containing unstructured text. The preprocessing operations of 762 generate one or more data outputs to be fed as data input into a second embedding layer 764. The second embedding layer 764 subsequently generates one or more translation of the patient data 782 into one or more vectors that are stored in a Patient Data Vector database 766.

In many implementations, the extractor algorithm employs at least one NLP Semantic Ranking operation 755 using data inputs obtained from the Z-Code Vector database 746 and Patient Data Vector database 766 to generate at least one Upper Threshold Result 748 and/or at least one Lower Threshold Result 768, wherein the upper and lower thresholds are determined by the semantic ranking. The NLP Semantic Ranking operation 755 enables the extractor algorithm to discern and extract relevant SDoH information from the patient data 782. Subsequently, the extracted SDoH information may undergo one or more reranking process whereby Upper Threshold Results 748 may be filtered by a first filter 750 to exclude false positives. Similarly, Lower Threshold Results 768 may be filtered by a second filter 790 to include one or more false negatives. In many implementations, the first and second filters 750, 790 then generate at least one filtered, final result 770 that identifies SDoH-related phrases from the patient data 782. The extractor algorithm may incorporate a human feedback operation 772 to at least one final result 770 as well as a fine-tuning stage 774 in which the model is continually refined. Fine tuning stage 774 iteratively generates one or more data sets that are subsequently stored either stored in an Exclude Queries Vector database 752 or stored in an Include Queries Vector database 752 and later used as input data for further refining of final result 770. The extraction algorithm may incorporate the iterative feedback mechanism to continually refine the model based on human feedback to enhance or improve performance over time.

The generation of a customizable SDoH plan can include adaptation (e.g., fine-tuning and transfer learning) of an existing LLM according to many implementations. One or more corpus of SDoH data can be split equally for training, development, and testing respectively. Testing and evaluation include the validation of the fine-tuned model on an external dataset (i.e., separate from the training data) and evaluation of the model's performance for a specific task. Model evaluation can involve the computation of metrics including accuracy, precision, recall, and/or an F1-score.

The disclosed LLM may be, for example, an autoregressive, masked, multimodal, contrastive, variant, modified, derivatives, present, or future version of T5, mT5, Auto-GPT, GPT-3, GPT-4, TO, CPM-2, Codex, ERNIE 3.0, ERNIE 3.0 Titan, Jurassic-1, HyperCLOVA, Gopher, GLaM, LaMDA, WebGPT, OPT-IML, mTO, Galactica, GLM, OPT, UL2, Tk-Instruct, GPT-NeoX-20B, CodeGen, MT-NLG, Chinchilla, PaLM, PaLM 2 Alexa™, Sparrow, U-PaLM, Flan-U-PalM, Flan-T5 XXL Flan-TX XL and XXL, BLOOM, ChatGPT, LlaMA, LLMA-2, MPT, Goat, Koala, WizardLM, Vicuna, Alpaca, Claude, Bard, BART, PaLM 2, Med-PaLM 2, BERT, ROBERTa, DeBERTa, DistilBERT, XLM, XLNet, M2M100, LUKE, ELECTRA, Longformer, etc., or combinations thereof.

In many implementations, one or more domain-specific models (e.g., the SDoH domain) are incorporated for tailored healthcare responses and task automation for routine tasks like setting patient reminders, scheduling appointments, or filling out enrollment forms. In various implementations, an ensemble of trained models and task-specific assignments may be configured to ensure the one or more models work collaboratively, contribute respective strengths, and to ensure that each task has the appropriate output. One or more frameworks (e.g., NIST's SP-1270) may be employed for said AI agent model bias mitigation and prevention ensuring robust training data and minimizing erroneous predictions.

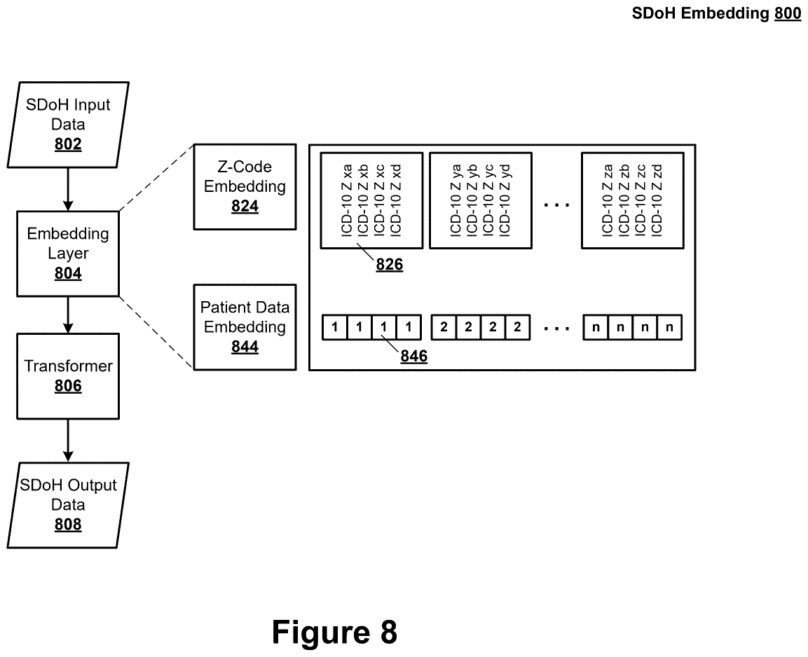

FIG. 8 is an architecture diagram 800 showing vector embedding of SDoH, leveraged by many implementations of the technology disclosed. SDoH data 802 may be processed, as input, through one or more embedding layers 804, and further processed by one or more transformer models 806, wherein said transformer(s) 806 subsequently produce one or SDoH prediction outputs 808. In some implementations, embedding layers 804 comprise one or more contextualized pre-trained embedding frameworks. In other implementations, pre-trained embedding frameworks are contextualized to SDoH. In many implementations, the transformer 806 may be adapted to use one or more structured EHR modalities (e.g., ICD-10 Z-codes). In yet other implementations, the technology disclosed uses one or more code embedding layers that are projected from, e.g., code embedding layer 824 or patient data 844 layers. In various implementations, the said embedding layers may use one or more EHR data elements such as a set of ICD-10 Z-codes corresponding to a patient visit. In various implementations, embedding layer 804 may convert one or more Z-codes codes into low-dimensional representations 826 and patient data into low-dimensional representations 846. The prediction of one or more codes comprises the use of a masked language model to predict one or more masked code using sequential information from the forward and backward directions.

In one implementation, transformer 806 may be used to capture the relationship between codes and patient data as well as their representations. In other implementations, fine-tuning processes include one or more feed-forward neural network layers may be added to transformer 806 corresponding to one or more prediction tasks. In alternative implementations, one or more recurrent neural networks (RNNs) are included, configured for rolling over the output of token embeddings. In other implementations, model parameters may be loaded and initialized from a pre-trained transformer 806 and the parameters respective to one or more predictive tasks are updated using a training algorithm such as gradient descent and other alternatives that are readily recognizable to a user skilled in the art.

The one or more predictive models can be trained to perform tasks including Health Disparity Identification (e.g., a minority patient has not been prescribed pain management medication when they should have, patients who live in rural areas have fewer orders for a mammography and a higher prevalence of late-stage breast cancer, etc.), identification of unconscious or implicit bias that result in differences in care amongst various populations, accurate prediction of social needs (i.e., Barriers to Care) and risk of chronic conditions (i.e., Clinical Indicators), identification of patients with similar attributes (e.g., demographic, clinical, etc.) and/or social needs (e.g., poverty, food insecurity, etc.) corresponding to certain chronic conditions or diseases, correlation of social needs to a future risk of chronic diseases or complications (e.g., address patient's transportation barrier to care, otherwise there is a high risk of future Emergency Room visit(s) and complications with diabetes due to missed medication refills). The predictive tasks may further comprise the identification of a patient's strength, social needs, risks, gaps in care, etc., and combinations thereof.

In many implementations, the disclosed AI models generate one or more recommendations for service needs from one or more patient stated goals or preferences. These predictive tasks may enable the crafting of a SDoH care plan. The SDoH care plan may comprise the modification of a HL7 C-CDA Care Plan incorporating one or more SDoH-related or targeted patient-specific precision intervention. Targeted interventions can include one or more of a behavioral change, removal of barriers, providing transportation or housing, changing patient perceptions, providing social support, alleviating fear, prioritize social needs, identifying local resources to address said social needs, etc., and combinations thereof. The predictive tasks may be executed by the generative AI agent 168 of FIG. 1 such as the use of prompt engineering for accurate generation of the SDoH care plan. The generative AI agent 168 may generate one or more Health Equity Alerts such as confirmed or possible barriers to care, missing patient information, health alerts, health risks, etc., and combinations thereof. The AI agent 168 can also generate one or more prescriptive instructions and/or recommendations for clinical decision-making and interventions.

### Autonomous Care Navigator SaaS Platform and Graphical User Interface

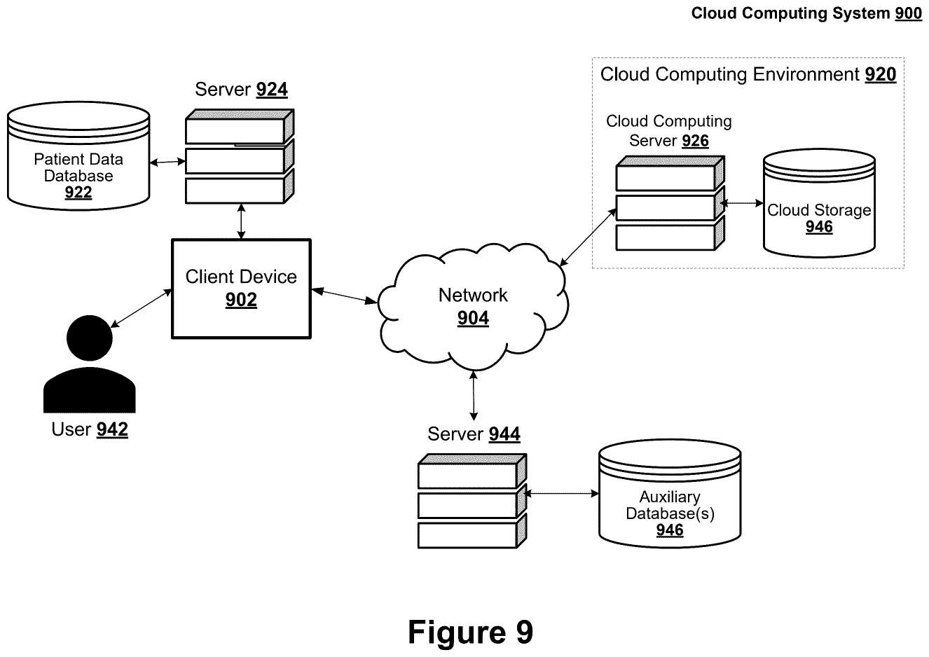

FIG. 9 is an architectural diagram 900 of a SaaS platform that can be leveraged in clinical decision-making. The architecture includes a client device connected to a cloud computing environment through a communication channel, e.g., a local area or wide area network interface. A user 942 such as a clinician interacts with a client computing device 902 that communicates with a server 924. The server 924 may be configured to communicate electronically with one or more databases 922, such as an EHR database. In certain implementations, the SaaS platform further includes one or more external electronic health or medical record servers 944 and/or auxiliary databases 946. In some implementations, the external server 944 and/or auxiliary database 946 comprises one or more of a third-party data server, third-party payor server, government server, and so on. These data server sources can be accessible via one or more APIs or cloud platforms. Example data sources may include CDC Behavioral Risk Factor Surveillance System, National Vital Statistics System, Substance Abuse and Mental Health Services Administration.

One or more components of the platform of diagram 900 may be communicate with a cloud computing environment via a telecommunication network 904. The telecommunication network 904 may comprise a LAN, WAN, wireless network, cellular network, the Internet, and other alternatives readily recognizable to a user skilled in the art. A cloud computing environment 920 comprises one or more servers 926 configured to operably engaged electronically with one or more databases 946, according to many implementations.

In various implementations, the cloud computing environment may perform or provide one or more services/functions generally understood and referred to as “cloud computing,” “on-demand computing,” “software as a service (SaaS),” “platform computing,” “cloud services,” “data centers,” and the like. The term “cloud” generally encompasses a collection of hardware and software that forms a shared pool of configurable computing resources (e.g., networks, servers, storage, applications, services, etc.) suitably provisioned to provide on-demand self-service, network access, resource pooling, elasticity, and measured service, among other features. In various implementations, the cloud computing server 816 may comprise a cloud-based control service server or a secured HIPAA-compliant remote application World Wide Web (“Web”) server.

In many implementations, the telecommunication network 904 comprises one or more network interfaces to enable one or more real-time interoperable data transmission using one or more data standards (e.g., FHIR, HL7). In various implementations, the SaaS platform may provide one or more real-time patient specific insights or information from one or more outputs generated by autonomous care navigator and/or the generative AI agent. In various implementations, the generated outputs are presented to user 942 via a dashboard or graphical user interface (GUI), e.g., via a display of client computing device 902. In an alternative implementation, the client computing device 902 is a smartphone configured with a mobile application for displaying non-limiting insight information. In various implementations, the GUI or mobile application may contain one or more elements depicting text, chart, graphics, video, or charts, among others. The said dashboard within GUI may be configured to enable healthcare provider user 942 to access the SaaS platform via a Web portal.

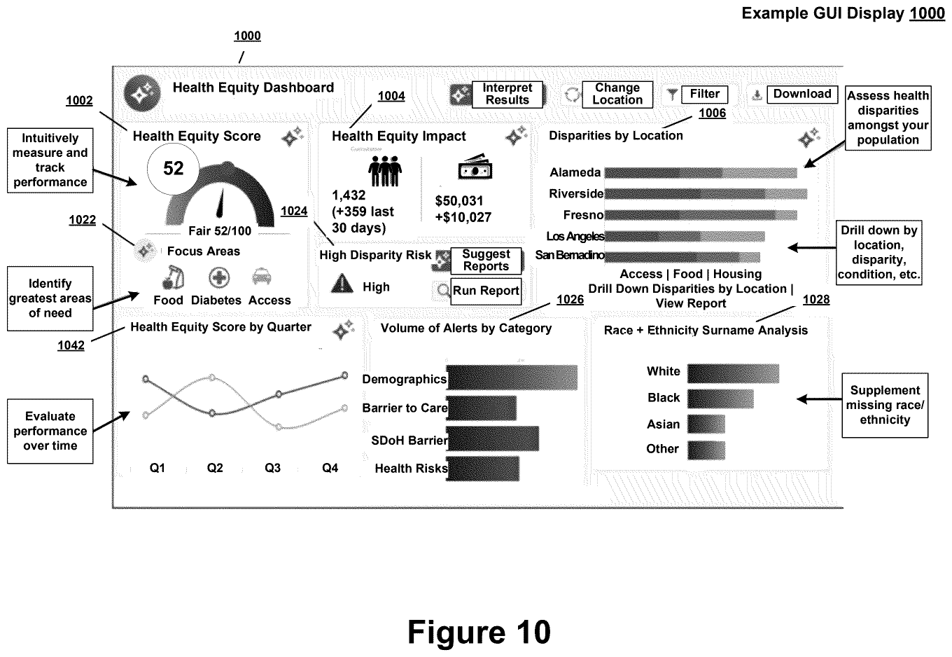

FIG. 10 is an example GUI dashboard associated with the SaaS platform of FIG. 10. Dashboard 1000 may comprise one or more graphical representations of insights, including a Health Equity Score 1002, Health Equity Score over time 1042 (i.e., historical trends in Health Equity Score), Focus Areas 1022, Health Equity Impact 1004, Volume of Alerts by Category 1026, Health Disparity Risk 1024, Disparity by Location 1006, Race and Ethnicity Surname Analysis 1028, and so on. Health Equity Score 1002 is a quantification of health disparities, including insights into the inequities that exist in healthcare access and health outcomes among various demographic groups. Example outputs may include a numeric score or a qualitative metric such as a letter grade or other representation of a scale, e.g., “excellence, good, needs improvement, poor, very poor,” and so on. In one example, the Health Equity Score 1002 may enable a healthcare provider to intuitively measure and track performance. In another example, the Focus Area 1022 may enable a healthcare provider to identify the greatest areas of need. In another example, the Health Equity Score per time 1042 allows a healthcare provider to evaluate performance over time. In yet another example, the Health Disparity Risk 1024 is determined as the likelihood or probability of disadvantaged groups facing a higher risk of contracting diseases or experiencing negative health outcomes. In various implementations, additional scores not listed with reference to FIG. 10 are provided to a user. A Health Risk Score (not displayed) assesses the overall health risk faced by a population, which includes the prevalence of chronic conditions, prevention measures, and unhealthy behaviors. SDoH Risk is a measure that predicts the likelihood of the population to contract disease based on social and economic factors. A user may use the Disparity by Location insight 1006 to assess health disparities among a patient population as well as the ability to drilldown by location, by disparity, condition, demographics, and so on.

In various implementations of the technology disclosed, a score such as those described above with reference to FIG. 10 may be derived and quantified from one or more parameters or factors relevant to each respective score. In one example, Health Equity Score 1002 may be derived to quantify a state in which an individual or patient has a fair and just opportunity to attain his or her highest level of health. The calculation of said Health Equity Score may comprise a combination of major units of analysis for measuring or quantifying health equity. In some implementations, the unit of analysis may comprise the quality of care given to the individual within a hospital, care facility, or a hospital service area (HSA). One example factor may comprise access, whereby a hospital's patient population is based on the demographics of HSA and the value of a score may be further based on racial, gender, and income disparities. With respect to access for a low-income patient, for example, the ratio of Medicaid beneficiaries in the HSA to civilian noninstitutionalized population should be comparable to the ratio of Medicaid discharges to total discharges.

Depending upon the nature of the treatments/services of a hospital, the racial, gender, and income level disparities in all major services/treatments contribute to the health equity score. Another factor may comprise health outcome whereby the outcome of a treatment/service may be independent of race/gender/income etc. There may be multiple factors, like carelessness, inexperienced staff, negligence, or biasedness. The outcome may be measured by the number of readmissions after the first discharge. An important measure or factor may comprise SDoH which accounts for most health outcomes. Hospitals, employees, and associated healthcare providers may positively intervene to improve or attain a high health equity score compared to a baseline or reference group. Charity care may be another measure or factor. Despite insurance programs, for example Care Act and Medicaid, there are still uninsured patients. The Charity care measure may require that ratio of charity to the total cost of a hospital should be comparable to the ratio of uninsured people to the total population in each HSA. Yet another factor may be Ambulatory care sensitive conditions (ACSCs) which are acute or chronic health issues that lead to potentially preventable hospitalizations when not treated in the outpatient primary care setting.

Effective outpatient care focusing on the HSA may prevent or at least reduce the risk of hospitalization. Therefore, a health equity score may comprise one or measure, including but not limited to, demographic, level of income (e.g., low), healthcare treatments, procedures, or services, health care outcomes (e.g., readmission), ACSCs, Charity, among others. In various implementations, a health equity score may comprise the number of tests per score and a formula may be derived for each said measure. For example, the demographic measure formula may comprise the multiplication of one or more ratio of race census (e.g., white, non-white) data, patient population (e.g., Medicare, Medicaid) within an HSA or county. In one implementation, one or more said scores are computed without requiring data from a specific healthcare provider client user of the platform. A health equity score may be measured using data from one or more of external database sources, including but not limited to, Dartmouth Atlas Hospital Tracking (Real), CMS Hospital Service Area (Real), UDS Mapper Zip Code to ZCTA-crosswalk, Census Racial Data based on ZCTA (Real), CMS Synthetic Patient Data OMOP, Future Data Sets, among others.

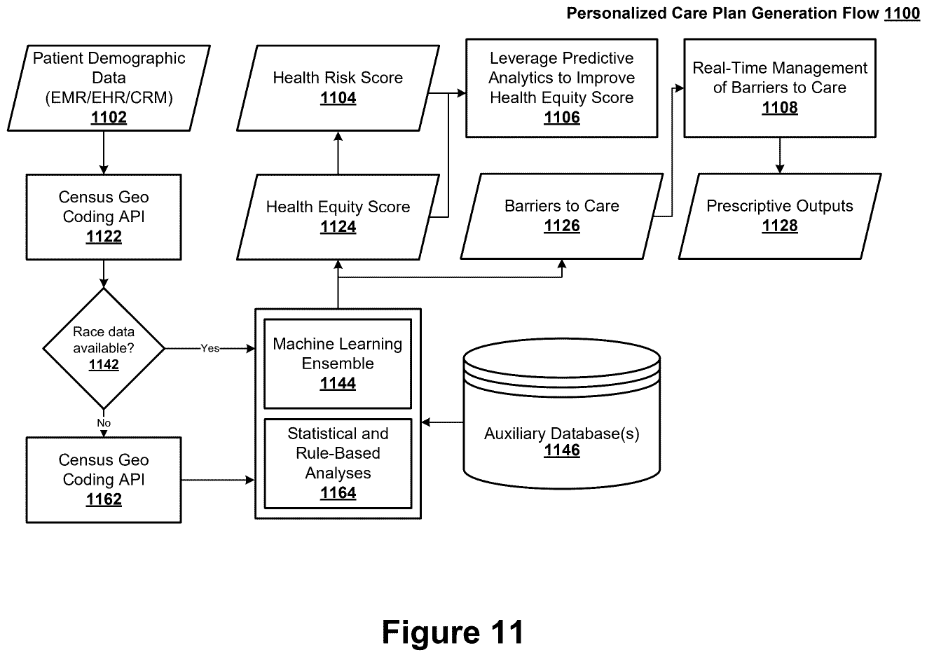

FIG. 11 is a block diagram of a process for generating information and presentation of generated information to a user via the GUI of FIG. 10. In a first retrieval operation, a patient demographic 1102 is retrieved for at least one EMR/EHR/CRM. A next operation 1122 comprises the census coding/retrieving of geolocation data for the patient, e.g., via an API. Next, in a decision operation 1142, a determination is made whether race information is available for the patient. If race information is not available, then geocoding 1162 is performed leveraging Bayesian inference and a patient surname. The input is provided into an Analytics/Predictive Engine. If race information is available, the race information can also serve as an input into Analytics/Predictive Engine. In various implementations, Analytics/Predictive Engine can include one or more analytics models, including but not limited to an AI/ML model 1144, additional statistical methods 1164, and other rule-based models.

In various implementations, the AI/ML model 1144 is an architecture as previously described above with reference to FIGS. 1-10. In many implementations, Analytics/Predictive Engine processes one or more data inputs in combination with one or more retrieved data from external data sources to generate, e.g., a Health Equity Score 1124 or a Health Risk Score 1104. In other implementations, the Analytics/Predictive Engine may generate one or more Barriers to Care 1126 in which the platform may generate information for the real-time management 1108 of Barriers to Care 1126. These steps may also comprise one more inputs obtained from database 1146 enabling an ability to merge outside data insights with patient data. The overall process may provide actionable and prescriptive directions 1128 via Heath Equity Dashboard 1000 of FIG. 10.

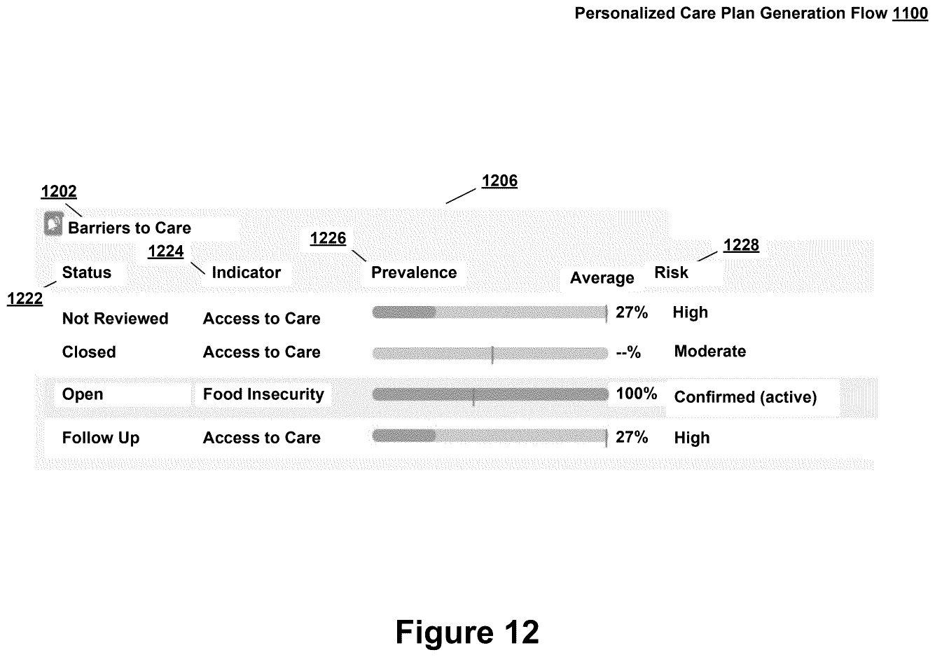

FIG. 12 is a second example GUI associated with the SaaS platform of FIG. 9. In one example implementation, a graphical output 1206 of Barriers to Care 1202 may comprise elements such as one or more of a Status 1222, Indicator 1224, Prevalence 1226, and Risk 1228. Status 1222 outputs may comprise one or more status including but not limited to Not Reviewed, Closed, Open, Follow Up, and so on. Indicator 1224 outputs may comprise one or more statuses or alerts including but not limited to Access to Care, Food Insecurity, among others. Prevalence 1226 outputs may include one or more average percentages (range from 0-100%). Risk 1228 outputs may include non-limiting risk levels such as High, Moderate, Low, among others.

Many implementations comprise a SaaS platform product implemented in electronic hardware, computing device, or software instructions stored and executable from one or more non-transitory storage medium located locally a client device 902 of FIG. 9 or mobile computing platform (e.g., via a smart phone), a healthcare provider or hospital computing server 924, or remotely on a cloud server 926 of FIG. 9 or cloud service. An exemplary computing device may comprise one or more processors, memory storage (e.g., RAM, ROM, etc.) devices, I/O devices, buses, and display.

### Computer System

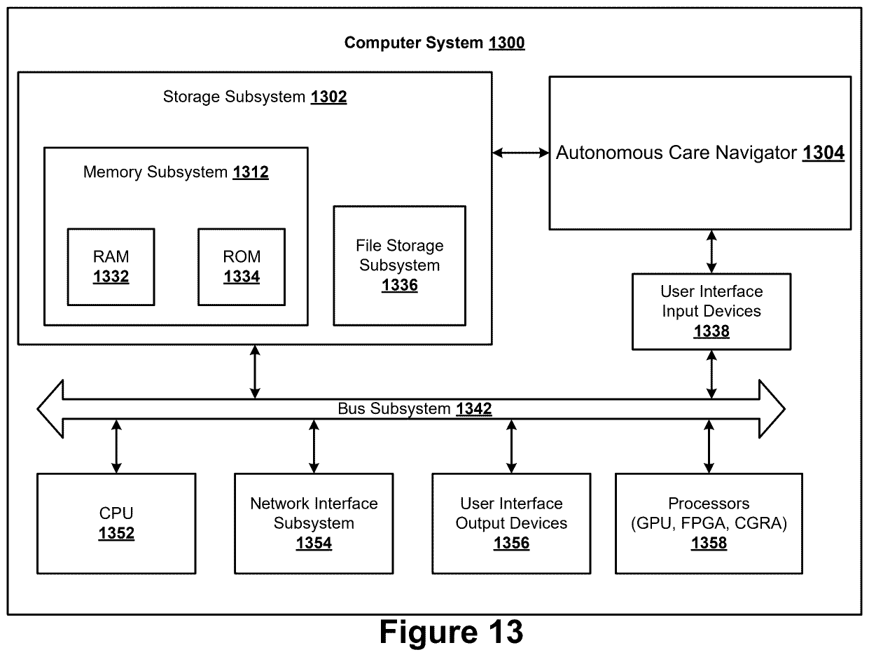

FIG. 13 is a simplified block diagram of a computer system 1300 that can be used for an automated workflow for social determinants of health data management, according to one implementation of the disclosed technology.

Computer system 1300 includes at least one central processing unit (CPU) 1352 that communicates with a number of peripheral devices via bus subsystem 1342 and autonomous care navigator 1304, as described herein. These peripheral devices can include a storage subsystem 1302 including, for example, memory devices and a file storage subsystem 1336, user interface input devices 1338, user interface output devices 1356 and a network interface subsystem 1354. The input and output devices allow user interaction with computer system 1300. Network interface subsystem 1354 provides an interface to outside networks, including an interface to corresponding interface devices in other computer systems. Autonomous care navigator 1304 is communicably linked to the storage subsystem 1302 and the user interface input devices 1338.

User interface input devices 1338 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include the possible types of devices and ways to input information into computer system 1300.

User interface output devices 1356 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include the possible types of devices and ways to output information from computer system 1300 to the user or to another machine or computer system.

Storage subsystem 1302 stores programming and data constructs that provide the functionality of the of the modules and methods described herein. Subsystem 1358 can be graphics processing units (GPUs) or field-programmable gate arrays (FPGAs).

Memory subsystem 1312 used in the storage subsystem 1302 can include a number of memories including a main random-access memory (RAM) 1332 for storage of instructions and data during program execution and a read only memory (ROM) 1334 in which fixed instructions are stored. A file storage subsystem 1336 can provide persistent storage for program and data files, and can include a hard disk drive, a floppy disk drive along with associated removable media, a CD-ROM drive, a DVD drive, a Blu-ray drive, an optical drive, or removable media cartridges. The modules implementing the functionality of certain implementations can be stored by file storage subsystem 1336 in the storage subsystem 1336, or in other machines accessible by the processor.

Bus subsystem 1342 provides a mechanism for letting the various components and subsystems of computer system 1300 communicate with each other as intended. Although bus subsystem 1342 is shown schematically as a single bus, alternative implementations of the bus subsystem can use multiple busses.

Computer system 1300 itself can be of varying types including a personal computer, a portable computer, a workstation, a computer terminal, a network computer, a television, a mainframe, a server farm, a widely distributed set of loosely networked computers, or any other data processing system or user device. Due to the ever-changing nature of computers and networks, the description of computer system 1300 depicted in FIG. 13 is intended only as a specific example for purposes of illustrating the preferred embodiments of the present invention. Many other configurations of computer system 1300 are possible having more or fewer components than the computer system depicted in FIG. 13.

### Particular Implementations

We describe various implementations of a method for generating a personalized care plan for a patient based on social determinants of health (SDoH). The method further comprises pre-processing unstructured patient data corresponding to a patient, received from a plurality of disparate patient data sources, wherein the pre-processing includes parsing the unstructured patient data for clinically relevant information, extracting features within the parsed clinically relevant information, wherein at least one feature is based on supplemental data from a clinical database, a SDoH dataset, or a SDoH coding dictionary, and converting the unstructured patient data into structured patient data including the extracted features. The method further includes processing the structured patient data, using a SDoH machine learning model, wherein the SDoH machine learning model is pre-trained to generate SDoH output data including at least one of: a barrier to care, a disease risk factor, a discrepancy in the structured patient data, a risk score, and a recommended SDoH intervention. The method further includes creating a personalized care plan for the patient, based on the SDoH output data, including a personalized resource recommendation, wherein the personalized resource recommendation: (i) identifies an action plan responsive to an identified barrier to care, and (ii) is constrained by a set of rules limiting the personalized resource recommendation to resources that are compatible with a geographic location, a socioeconomic status, or a disability status of the patient. The method further includes displaying to a user, via a user interface of an autonomous care navigator, the personalized care plan, wherein the user interface is configured to receive user feedback including updated patient data or progress data related to execution of the personalized care plan, and updating the personalized care plan based on the user feedback, wherein a feedback loop for the SDoH machine learning model leverages the user feedback to refine the SDoH output data, and the personalized care plan is updated in dependence on the refined SDoH output data.

In one implementation, the plurality of disparate patient data sources includes one or more of: an electronic health record, a social work documentation, a patient screening assessment, and a billing history. In another implementation, the plurality of disparate patient data sources includes one or more of a text format, an audio format, and a video format. In yet another implementation, the parsed clinically relevant information includes at least one of: a patient identity, a demographic, a disease, a diagnostic code, a residence, a medical encounter, a clinical risk factor, and a clinical event. In some implementations, extracting the features within the parsed clinically relevant information further includes obtaining an extracted feature from a particular data source within the plurality of disparate patient data sources and converting a raw data format of the extracted feature from the particular data source to a standardized data format that is consistent across the plurality of disparate patient data sources, transforming an extracted feature from an unstructured format into a structured format that is compatible with input requirements of the trained machine learning model, or constructing a feature from (i) an extracted feature and (i) at least one additional extracted feature from the parsed clinically relevant information or a supplemental data source, wherein the supplemental data source is a clinical database, a SDoH dataset, or a clinical coding dictionary.

One disclosed method further includes extraction of an ICD-10 Z-code from the parsed clinically relevant information and mapped to a supplemental SDoH coding dictionary to construct an SDoH attribute feature. In another disclosed method, a patient surname is extracted from the parsed clinically relevant information and mapped to an ethnicity dataset to construct an ethnicity feature. In other disclosed methods, a residence is extracted from the parsed clinically relevant information and mapped to a geocoding database to construct a resource access attribute characterizing a nutrition access level, a transportation access level, or a healthcare access level.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A method implementation of the technology disclosed includes an autoencoder trained as an SDoH machine learning model. Another method implementation includes a name-entity recognition model as an SDoH machine learning model. Another method implementation includes a large language model as an SDoH machine learning model. In one implementation, the method further includes post-processing the SDoH output data using a statistical analysis, wherein the statistical analysis comprises comparing the SDoH output data to population data from a clinical database, identifying a health pattern over time for the patient based on a trend analysis evaluating SDoH output data corresponding to a plurality of time points, computing an aggregated risk metric from a plurality of SDoH outputs, and converting quantitative SDoH output data to a qualitative metric, wherein the conversion further includes categorizing the quantitative SDoH output data based on binning, clustering, or a classification based on a comparison of a quantitative SDoH output value to a pre-defined threshold value.

Some implementations include processing the structured patient data by an ensemble of SDoH machine learning models, wherein each SDoH machine learning model of the ensemble generates at least one respective SDoH output, and post-processing the SDoH outputs of the ensemble, including statistical analysis of a combination of two or more respective SDoH outputs from the ensemble to generate subsequent SDoH data.

Creating a personalized care plan can further include curating a selection of SDoH data from the SDoH output data and statistical analysis of the SDoH output data, wherein the selected SDoH data represents an overview of a patient health status and SDoH factors impacting the patient health status, matching the identified barrier to care for the patient to one or more appropriate SDoH interventions, wherein an appropriate match is determined based on at least one validated clinical data source, searching at least one of a curated database and an internet search engine, using a generative artificial intelligence agent, to identify patient resources providing at least one matched SDoH resource, wherein the searching is constrained by a pre-determined distance threshold from a residence of the patient, and ranking the identified patient resources in dependence upon the set of rules limiting the personalized resource recommendation to determine a best fit resource as the personalized resource recommendation.

In some implementations, the disclosed the autonomous care navigator enables the user to drill down on a particular element of the displayed personalized care plan within the user interface to view additional data about an associated SDoH output or statistical analysis used to generate the particular element. Another implementation of the technology disclosed comprises the autonomous care navigator compiling SDoH output data and personalized care plans for a plurality of patients and displaying, via the user interface, summary statistics characterizing population-level SDoH data for the plurality of patients.

A system implementation of the technology disclosed relates to an autonomous care navigator configured to generate a personalized care plan for a patient based on social determinants of health (SDoH), the autonomous care navigator comprising a processor and memory coupled to the processor. The autonomous care navigator further comprises a data pre-processor configured for pre-processing unstructured patient data corresponding to a patient, received from a plurality of disparate patient data sources, wherein the pre-processing includes parsing the unstructured patient data for clinically relevant information, extracting features within the parsed clinically relevant information, wherein at least one feature is based on supplemental data from a clinical database, a SDoH dataset, or a SDoH coding dictionary, and converting the unstructured patient data into structured patient data including the extracted features. The autonomous care navigator also includes a SDoH machine learning model, wherein the SDoH machine learning model is pre-trained to process the structured patient data and to generate SDoH output data including at least one of a barrier to care, a disease risk factor, a discrepancy in the structured patient data, a risk score, and a recommended SDoH intervention. The autonomous care navigator also includes a generative AI agent, wherein the generative AI agent is pre-trained to create a personalized care plan for the patient, based on the SDoH output data, including a personalized resource recommendation, wherein the personalized resource recommendation identifies an action plan responsive to an identified barrier to care, and is constrained by a set of rules limiting the personalized resource recommendation to resources that are compatible with a geographic location, a socioeconomic status, or a disability status of the patient. The autonomous care navigator also includes a graphical user interface configured for (i) displaying, to a user, the personalized care plan, and (ii) receiving user feedback including updated patient data or progress data related to execution of the personalized care plan and transmitting the received user feedback to the SDoH machine learning model. A feedback loop for the SDoH machine learning model leverages the user feedback to refine the SDoH output data, and the generative AI agent updates the personalized care plan in dependence on the refined SDoH output data.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

Other implementations may include a non-transitory computer readable storage medium storing instructions executable by a processor to perform functions of the system described above. Yet another implementation may include a method performing the functions of the system described above.

Various implementations of the technology disclosed include any of the methods described herein, wherein the feedback loop further comprises receiving, via the user interface, a user feedback input, wherein the user feedback input is a new medical encounter, a new medication, a new diagnosis, a lifestyle modification, a health status descriptor, or the progress data related to the execution of the personalized care plan, providing, as input to the SDoH machine learning model, previously generated SDoH output data for the patient, the personalized care plan, and the user feedback, to generate updated SDoH output data, updating the personalized care plan for the patient based on the updated SDoH output data, and displaying to the user, via the user interface of the autonomous care navigator, the updated personalized care plan.

As indicated above, all the system features are not repeated here and should be considered repeated by reference.

In one implementation of the technology disclosed, the user is the patient, a healthcare provider for the patient, a case worker or social worker for the patient, a caregiver for the patient, a healthcare payor professional, and/or a regulatory and compliance professional.

Each of the features discussed in this particular implementation section for the first system implementation apply equally to this method implementation. As indicated above, all the system features are not repeated here and should be considered repeated by reference.

Other implementations may include a non-transitory computer readable storage medium storing instructions executable by a processor to perform a method as described above. Yet another implementation may include a system including memory and one or more processors operable to execute instructions, stored in the memory, to perform a method as described above.

The technology disclosed can be practiced as a system, method, or article of manufacture. One or more features of an implementation can be combined with the base implementation. Implementations that are not mutually exclusive are taught to be combinable. One or more features of an implementation can be combined with other implementations.

While the technology disclosed is disclosed by reference to the preferred implementations and examples detailed above, it is to be understood that these examples are intended in an illustrative rather than in a limiting sense. It is contemplated that modifications and combinations will readily occur to those skilled in the art, which modifications and combinations will be within the spirit of the innovation and the scope of the following claims.

1. A computer-implemented method for generating a personalized care plan for a patient based on social determinants of health (SDoH), the computer-implemented method comprising:
pre-processing unstructured patient data corresponding to a patient, received from a plurality of disparate patient data sources, wherein the pre-processing includes:
parsing the unstructured patient data for clinically relevant information,
extracting features within the parsed clinically relevant information, wherein at least one feature is based on supplemental data from a clinical database, a SDoH dataset, or a SDoH coding dictionary, and
converting the unstructured patient data into structured patient data including the extracted features;
processing the structured patient data, using a SDoH machine learning model, wherein the SDoH machine learning model is pre-trained to generate SDoH output data including at least one of: a barrier to care, a disease risk factor, a discrepancy in the structured patient data, a risk score, and a recommended SDoH intervention;
creating a personalized care plan for the patient, based on the SDoH output data, including a personalized resource recommendation, wherein the personalized resource recommendation: (i) identifies an action plan responsive to an identified barrier to care, and (ii) is constrained by a set of rules limiting the personalized resource recommendation to resources that are compatible with a geographic location, a socioeconomic status, or a disability status of the patient;
displaying to a user, via a user interface of an autonomous care navigator, the personalized care plan, wherein the user interface is configured to receive user feedback including updated patient data or progress data related to execution of the personalized care plan; and
updating the personalized care plan based on the user feedback, wherein a feedback loop for the SDoH machine learning model leverages the user feedback to refine the SDoH output data, and the personalized care plan is updated in dependence on the refined SDoH output data.

2. The computer-implemented method of claim 1, wherein the plurality of disparate patient data sources includes one or more of: an electronic health record, a social work documentation, a patient screening assessment, and a billing history.

3. The computer-implemented method of claim 1, wherein the plurality of disparate patient data sources includes one or more of a text format, an audio format, and a video format.

4. The computer-implemented method of claim 1, wherein the parsed clinically relevant information includes at least one of: a patient identity, a demographic, a disease, a diagnostic code, a residence, a medical encounter, a clinical risk factor, and a clinical event.

5. The computer-implemented method of claim 1, wherein extracting the features within the parsed clinically relevant information further includes:
obtaining an extracted feature from a particular data source within the plurality of disparate patient data sources and converting a raw data format of the extracted feature from the particular data source to a standardized data format that is consistent across the plurality of disparate patient data sources;
transforming an extracted feature from an unstructured format into a structured format that is compatible with input requirements of the trained machine learning model; or
constructing a feature from (i) an extracted feature and (ii) at least one additional extracted feature from the parsed clinically relevant information or a supplemental data source, wherein the supplemental data source is a clinical database, a SDoH dataset, or a clinical coding dictionary.

6. The computer-implemented method of claim 5, wherein an ICD-10 Z-code is extracted from the parsed clinically relevant information and mapped to a supplemental SDoH coding dictionary to construct an SDoH attribute feature.

7. The computer-implemented method of claim 5, wherein a patient surname is extracted from the parsed clinically relevant information and mapped to an ethnicity dataset to construct an ethnicity feature.

8. The computer-implemented method of claim 5, wherein a residence is extracted from the parsed clinically relevant information and mapped to a geocoding database to construct a resource access attribute characterizing a nutrition access level, a transportation access level, or a healthcare access level.

9. The computer-implemented method of claim 1, wherein the SDoH machine learning model is an autoencoder.

10. The computer-implemented method of claim 1, wherein the SDoH machine learning model is a name-entity recognition model.

11. The computer-implemented method of claim 1, wherein the SDoH machine learning model is a large language model.

12. The computer-implemented method of claim 1, further including post-processing the SDoH output data using a statistical analysis, wherein the statistical analysis comprises:
comparing the SDoH output data to population data from a clinical database;
identifying a health pattern over time for the patient based on a trend analysis evaluating SDoH output data corresponding to a plurality of time points;
computing an aggregated risk metric from a plurality of SDoH outputs; and
converting quantitative SDoH output data to a qualitative metric, wherein the conversion further includes categorizing the quantitative SDoH output data based on binning, clustering, or a classification based on a comparison of a quantitative SDoH output value to a pre-defined threshold value.

13. The computer-implemented method of claim 1, further comprising:
processing the structured patient data by an ensemble of SDoH machine learning models, wherein each SDoH machine learning model of the ensemble generates at least one respective SDoH output; and
post-processing the SDoH outputs of the ensemble, including statistical analysis of a combination of two or more respective SDoH outputs from the ensemble to generate subsequent SDoH data.

14. The computer-implemented method of claim 1, wherein creating the personalized care plan for the patient further includes:
curating a selection of SDoH data from the SDoH output data and statistical analysis of the SDoH output data, wherein the selected SDoH data represents an overview of a patient health status and SDoH factors impacting the patient health status;
matching the identified barrier to care for the patient to one or more appropriate SDoH interventions, wherein an appropriate match is determined based on at least one validated clinical data source;
searching at least one of a curated database and an internet search engine, using a generative artificial intelligence agent, to identify patient resources providing at least one matched SDoH resource, wherein the searching is constrained by a pre-determined distance threshold from a residence of the patient; and
ranking the identified patient resources in dependence upon the set of rules limiting the personalized resource recommendation to determine a best fit resource as the personalized resource recommendation.

15. The computer-implemented method of claim 1, wherein the autonomous care navigator enables the user to drill down on a particular element of the displayed personalized care plan within the user interface to view additional data about an associated SDoH output or statistical analysis used to generate the particular element.

16. The computer-implemented method of claim 1, further comprising the autonomous care navigator compiling SDoH output data and personalized care plans for a plurality of patients and displaying, via the user interface, summary statistics characterizing population-level SDoH data for the plurality of patients.

17. The computer-implemented method of claim 1, wherein the feedback loop further comprises:
receiving, via the user interface, a user feedback input, wherein the user feedback input is a new medical encounter, a new medication, a new diagnosis, a lifestyle modification, a health status descriptor, or the progress data related to the execution of the personalized care plan;
providing, as input to the SDoH machine learning model, previously generated SDoH output data for the patient, the personalized care plan, and the user feedback, to generate updated SDoH output data;
updating the personalized care plan for the patient based on the updated SDoH output data; and
displaying to the user, via the user interface of the autonomous care navigator, the updated personalized care plan.

18. The computer-implemented method of claim 1, wherein the user is the patient, a healthcare provider for the patient, a case worker or social worker for the patient, a caregiver for the patient, a healthcare payor professional, or a regulatory and compliance professional.

19. An autonomous care navigator configured to generate a personalized care plan for a patient based on social determinants of health (SDoH), the autonomous care navigator comprising a processor and memory coupled to the processor, the autonomous care navigator further comprising:
a data pre-processor configured for pre-processing unstructured patient data corresponding to a patient, received from a plurality of disparate patient data sources, wherein the pre-processing includes:
parsing the unstructured patient data for clinically relevant information,
extracting features within the parsed clinically relevant information, wherein at least one feature is based on supplemental data from a clinical database, a SDoH dataset, or a SDoH coding dictionary, and
converting the unstructured patient data into structured patient data including the extracted features;
a SDoH machine learning model, wherein the SDoH machine learning model is pre-trained to process the structured patient data and to generate SDoH output data including at least one of: a barrier to care, a disease risk factor, a discrepancy in the structured patient data, a risk score, and a recommended SDoH intervention;
a generative AI agent, wherein the generative AI agent is pre-trained to create a personalized care plan for the patient, based on the SDoH output data, including a personalized resource recommendation, wherein the personalized resource recommendation:
(i) identifies an action plan responsive to an identified barrier to care, and (ii) is constrained by a set of rules limiting the personalized resource recommendation to resources that are compatible with a geographic location, a socioeconomic status, or a disability status of the patient; and
a graphical user interface configured for (i) displaying, to a user, the personalized care plan, and (ii) receiving user feedback including updated patient data or progress data related to execution of the personalized care plan and transmitting the received user feedback to the SDoH machine learning model,
wherein a feedback loop for the SDoH machine learning model leverages the user feedback to refine the SDoH output data, and the generative AI agent updates the personalized care plan in dependence on the refined SDoH output data.

20. A non-transitory computer readable storage medium impressed with computer program instructions to generate a personalized care plan for a patient based on social determinants of health (SDoH), the instructions, when executed on a processor, implement the following operations:
pre-processing unstructured patient data corresponding to a patient, received from a plurality of disparate patient data sources, wherein the pre-processing includes:
parsing the unstructured patient data for clinically relevant information,
extracting features within the parsed clinically relevant information, wherein at least one feature is based on supplemental data from a clinical database, a SDoH dataset, or a SDoH coding dictionary, and
converting the unstructured patient data into structured patient data including the extracted features;
processing the structured patient data, using a SDoH machine learning model, wherein the SDoH machine learning model is pre-trained to generate SDoH output data including at least one of: a barrier to care, a disease risk factor, a discrepancy in the structured patient data, a risk score, and a recommended SDoH intervention;
creating a personalized care plan for the patient, based on the SDoH output data, including a personalized resource recommendation, wherein the personalized resource recommendation: (i) identifies an action plan responsive to an identified barrier to care, and (ii) is constrained by a set of rules limiting the personalized resource recommendation to resources that are compatible with a geographic location, a socioeconomic status, or a disability status of the patient;
displaying to a user, via a user interface of an autonomous care navigator, the personalized care plan, wherein the user interface is configured to receive user feedback including updated patient data or progress data related to execution of the personalized care plan; and
updating the personalized care plan based on the user feedback, wherein a feedback loop for the SDoH machine learning model leverages the user feedback to refine the SDoH output data, and the personalized care plan is updated in dependence on the refined SDoH output data.
